(19) United States
(12) Patent Application Publication
(10) Pub. No.: US 2023/0348694 A1
(43) Pub. Date: Nov. 2, 2023

(54) SHELF LIFE- AND TRANSPORT STABILIZERS FOR POLYMERIZABLE COMPOUNDS

(71) Applicant: BASF SE, Ludwigshafen am Rhein (DE)

(72) Inventors: Sylke HAREMZA, Ludwigshafen am Rhein (DE); Friedrich-Georg MARTIN, Ludwigshafen am Rhein (DE); Doris KREMZOW-GRAW, Ludwigshafen am Rhein (DE); Rebecca SURE, Ludwigshafen am Rhein (DE); Juergen ROMEIS, Ludwigshafen am Rhein (DE); Korwin SCHELKLE, Navi Mumbai (IN); Friederike FLEISCHHAKER, Ludwigshafen am Rhein (DE); Jan Niclas GORGES, Ludwigshafen am Rhein (DE)

(21) Appl. No.: 17/780,573

(22) PCT Filed: Nov. 23, 2020
(86) PCT No.: PCT/EP2020/083014

§ 371 Date: May 27, 2022

(30) Foreign Application Priority Data
Nov. 28, 2019 (EP) 19212189.5

(51) Int. Cl. C08K 5/3445; C08F 220/06; C08F 220/14; C08F 220/18

(52) U.S. Cl. CPC C08K 5/3445; C08F 220/06; C08F 220/14; C08F 220/1802; C08F 220/1804; C08F 220/1808

(57) ABSTRACT

A mixture comprising one or more compounds of the general formula (II)

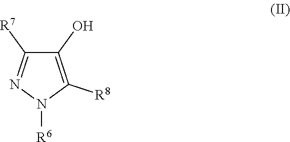

where
R⁶ is H, C₁ to C₂₀ alkyl, where C₁ to C₂₀ alkyl comprises straight-chain or branched C₁ to C₂₀ alkyl groups or cyclic C₃ to C₂₀ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
R⁷ is H, C₁ to C₂₀ alkyl, where C₁ to C₂₀ alkyl comprises straight-chain or branched C₁ to C₂₀ alkyl groups or cyclic C₃ to C₂₀ alkyl groups, phenyl, carboxylic esters having C₁ to C₈ alkyl, where C₁ to C₈ alkyl comprises straight-chain or branched C₁ to C₆ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl, and
R⁸ is H, C₁ to C₂₀ alkyl, where C₁ to C₂₀ alkyl comprises straight-chain or branched C₁ to C₂₀ alkyl groups or cyclic C₃ to C₂₀ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
and (meth)acrylic acid and/or C₁ to C₈ alkyl (meth)acrylates.

Chemical compounds that contain one or more ethylenically unsaturated groups have a pronounced tendency to free-radical polymerization. Such compounds are accordingly referred to as polymerizable compounds. The tendency of these compounds to free-radical polymerization means that they are used as monomers for the production of polymers, e.g. through intentional free-radical polymerization. However, the pronounced tendency to free-radical polymerization of these compounds is also a disadvantage in that undesired spontaneous free-radical polymerization can occur during storage and transport and also during chemical and/or physical processing, e.g. by distillation or rectification, particularly under the action of energy, e.g. heat and/or light. Such uncontrolled polymerizations can result in the gradual formation of polymer deposits, e.g. on heated surfaces, that necessitates removal of the polymer deposits and thus frequently to a reduction in operating times, or can even lead to explosions.

During the storage and transport and also during chemical and/or physical processing of ethylenically unsaturated compounds that have a tendency to free-radical polymerization or mixtures that contain such compounds, it is therefore customary to add substances that prevent or retard undesired spontaneous free-radical polymerization. Such substances are referred to generally and hereinafter as polymerization inhibitors or polymerization stabilizers.

Polymerization stabilizers may be used in the form of individual chemical compounds or mixtures of compounds. A polymerization stabilizer may be subject to particular requirements, depending on its field of use. For example, for a polymerization stabilizer to be suitable as a stabilizer for the transport and storage of ethylenically unsaturated compounds, it is important that the efficiency of the polymerization stabilizer, i.e. the extent of stabilization or inhibition of polymerization, can be regulated. Under the conditions of storage and/or transport of the ethylenically unsaturated compounds, the polymerization stabilizer should adequately prevent or retard undesired spontaneous free-radical polymerization, whereas the desired free-radical polymerization of the ethylenically unsaturated compounds should be possible under appropriate polymerization conditions without the need to first have to separate the polymerization stabilizer used during storage and/or transport. If the stabilizer used during storage and/or transport is not separated during the desired polymerization, it is important that this does not have an adverse effect during the desired polymerization or does not act unintentionally as a chain-transfer agent (for polymerization chain-transfer agents, see for example Ullmann's Encyclopedia of Industrial Chemistry, Polymerization Processes, 1. Fundamentals, 6 Free-Radical Polymerization, DOI 10.1002/14356007.21_305.pub3 or Ullmann's Encyclopedia of Industrial Chemistry, Polyacrylates, 4.1.1 Starting Materials, DOI 10.1002/14356007.a21_157.pub2).

In terms of global production volume, acrylic acid is without doubt one of the most important ethylenically unsaturated compounds. As standard, acrylic acid is stabilized against undesired spontaneous free-radical polymerization during storage and/or transport with 180 to 220 ppm by weight of hydroquinone monomethyl ether (MEHQ), based on the amount of acrylic acid. For adequate stabilization of acrylic acid against undesired spontaneous free-radical polymerization using MEHQ, it is necessary that oxygen is dissolved in the acrylic acid in sufficient amounts. Sufficient amounts of oxygen are generally dissolved in the acrylic acid when acrylic acid is stored and/or transported under an atmosphere containing 5 to 21 vol % of oxygen. The stabilization of acrylic acid by MEHQ during transport and/or storage is described for example in Acrylic Acid, A Summary of Safety and Handling, 4th edition 2013, 6.1 Instability and Reactivity—Polymerisation, and 7.1. Bulk Storage Facilities and Accessories—General Considerations. In the desired polymerization of acrylic acid, for example in the production of polyacrylates, the content of dissolved oxygen in the acrylic acid is reduced, which reduces the efficiency of MEHQ as a polymerization stabilizer, thereby allowing acrylic acid to undergo polymerization in the presence of MEHQ.

In addition to use as a polymerization stabilizer during storage and/or transport of acrylic acid, MEHQ is also used as a polymerization stabilizer during storage and/or transport of methacrylic acid, acrylic esters, methacrylic esters or mixtures comprising one or more of said compounds. Acrylic acid or methacrylic acid are referred to hereinafter as (meth)acrylic acid and the esters thereof referred to hereinafter as (meth)acrylic esters.

As a consequence of its breadth of use, MEHQ is one of the most important storage and/or transport stabilizers for polymerizable compounds in general and for (meth)acrylic acid and (meth)acrylic esters in particular.

Polymerizable compounds are stored in suitable, permanently installed containers such as storage tanks or other suitable storage vessels (see for example Acrylic Acid, A Summary of Safety and Handling, 4th edition 2013, 7 Bulk Storage Facilities and Accessories). Storage of polymerizable compounds in the respective containers is preferably for 1 minute or longer, more preferably for 10 minutes or longer, and particularly preferably for 60 minutes or longer. Although the duration of storage under appropriate conditions is theoretically unlimited, the duration of storage is generally reduced to a minimum for economic reasons. Storage of polymerizable compounds in the respective containers is preferably for not more than 180 days, more preferably for not more than 90 days, and particularly preferably for not more than 30 days. Particularly preferred ranges arise from the free combination of the abovementioned lower and upper ranges.

The transport of polymerizable compounds by ship, rail and/or truck is normally in suitable transportable containers such as tanks or other suitable vessels (see for example Acrylic Acid, A Summary of Safety and Handling, 4th edition 2013, 9 Safe Transport of Acrylic Acid). The transportable containers may of course also be permanently installed, for example tanks in ships or rail tankers. It is of course also possible for containers to be stored for a certain time in a particular place before the transport thereof somewhere else. The transport of polymerizable compounds may also be in pipelines or hoses, for example to a storage tank after purification of polymerizable compounds, during loading from the storage tank into a transportable container, and/or during loading from one transportable container into another. Transport of polymerizable compounds in the respective containers is preferably for a period of 10 seconds or longer, more preferably for 1 minute or longer, particularly preferably for 10 minutes or longer, and most preferably for 60 minutes or longer. Although the duration under appropriate conditions is theoretically unlimited, the duration is generally reduced to a minimum for economic and safety reasons. Transport of polymerizable compounds in the respective containers is preferably for not more than 180 days, more preferably for not more than 90 days, and particularly preferably for not more than 30 days. Particularly preferred ranges arise from the free combination of the abovementioned lower and upper ranges.

To reduce dependence on MEHQ, it was the object of the present invention to provide mixtures that comprise (meth)acrylic acid and/or C₁ to C₈ alkyl (meth)acrylates and one or more storage and/or transport stabilizers. The storage and/or transport stabilizers should ensure adequate stabilization of the (meth)acrylic acid and/or C₁ to C₈ alkyl (meth)acrylates against undesired free-radical polymerization. In addition, it would also be an advantage if, in the desired free-radical polymerization, there was no need to separate the storage and/or transport stabilizers from the mixture prior to the polymerization. The storage and/or transport stabilizers should therefore not act as chain-transfer agents and/or inhibitors in the desired free-radical polymerization.

In the context of the present invention, a compound is suitable as a storage and/or transport stabilizer for (meth)acrylic acid and/or C₁ to C₈ alkyl (meth)acrylates and does not act as a chain-transfer agent and/or inhibitor in the desired free-radical polymerization when, under comparable conditions, said compound
a) inhibits with an efficiency of 75 percent or more, based on the efficiency of an equimolar amount of MEHQ, the spontaneous free-radical polymerization of liquid acrylic acid stored under an atmosphere containing 5 to 30 vol % of oxygen
and
b) inhibits with an efficiency of 130 percent or less, based on the efficiency of an equimolar amount of MEHQ, the free-radical polymerization of liquid acrylic acid stored under an atmosphere containing 96 to 100 vol % of nitrogen and to which is added 1000 ppm to 15 000 ppm by weight of a polymerization initiator, comparable or similar polymers being obtained after the polymerization. The nitrogen content of the atmosphere is preferably 99 to 100 vol %.

In a) and b), MEHQ is in each case used in amounts of 10 ppm to 250 ppm by weight, preferably in an amount of 35 ppm to 45 ppm by weight. Values in ppm by weight are based on the amount of liquid acrylic acid used.

Comparable or similar polymers are obtained when the polymers have largely identical weight averages (measured according to the same GPC method). Largely identical weight averages are present when the weight averages are 20 percent or less apart.

Suitable as polymerization initiators are polymerization initiators known to those skilled in the art that are customarily used in the polymerization of (meth)acrylic acid (see for example The Chemistry of Radical Polymerisation, 2nd edition, 2005, pages 49 to 166).

The object is achieved by a ‘mixture comprising one or more compounds of the general formula (II)

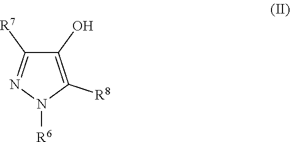

where
R⁶ is H, C₁ to C₂₀ alkyl, where C₁ to C₂₀ alkyl comprises straight-chain or branched C₁ to C₂₀ alkyl groups or cyclic C₃ to C₂₀ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
R⁷ is H, C₁ to C₂₀ alkyl, where C₁ to C₂₀ alkyl comprises straight-chain or branched C₁ to C₂₀ alkyl groups or cyclic C₃ to C₂₀ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl, and
R⁸ is H, C₁ to C₂₀ alkyl, where C₁ to C₂₀ alkyl comprises straight-chain or branched C₁ to C₂₀ alkyl groups or cyclic C₃ to C₂₀ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
and (meth)acrylic acid and/or C₁ to C₈ alkyl (meth)acrylates.
C₁ to C₈ alkyl comprises straight-chain or branched C₁ to C₈ alkyl groups or cyclic C₃ to C₈ alkyl groups. Examples of C₁ to C₈ alkyl are methyl, ethyl, n-propyl, isopropyl, n-butyl, sec-butyl, isobutyl, tert-butyl, n-pentyl, 2-methylbutyl, 3-methylbutyl, cyclopentyl, n-hexyl, cyclohexyl, 2-methylpentyl, 3-methylpentyl, 4-methylpentyl, 3,3-dimethylbutyl, 2-ethylbutyl, n-heptyl, 2-methylhexyl, 2-ethylpentyl, 4-methylcyclohexyl, n-octyl, isooctyl or 2-ethylhexyl.

The present invention further provides for the use of one or more compounds of the general formula (II) as storage and/or transport stabilizers for (meth)acrylic acid and/or C₁ to C₈ alkyl (meth)acrylates.

The present invention additionally provides a method for stabilizing (meth)acrylic acid and/or C₁ to C₈ alkyl (meth)acrylates during transport and/or storage through the use of one or more compounds of the general formula (II).

The present invention likewise provides a method for polymerizing (meth)acrylic acid and/or C₁ to C₈ alkyl (meth)acrylates in the presence of one or more compounds of the general formula (II).

The mixture according to the invention is a liquid under standard conditions.

### DESCRIPTION

In compounds of the general formula (I),

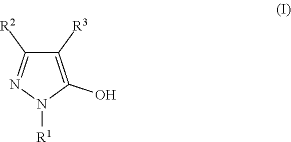

R¹ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R² is H, C₁ to C₂₀ alkyl, where C₁ to C₂₀ alkyl comprises straight-chain or branched C₁ to C₂₀ alkyl groups or cyclic C₁ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl, and
R³ is H, C₁ to C₂₀ alkyl, where C₂ to C₂₀ alkyl comprises straight-chain or branched C₂ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl.

In preferred compounds of the general formula (I),
R¹ is H, C₁ to C₆ alkyl, where C₁ to C₆ alkyl comprises straight-chain or branched C₁ to C₆ alkyl groups or cyclic C₃ to C₆ alkyl groups, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₁ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R² is H or C₁ to C₆ alkyl, where C₁ to C₆ alkyl comprises straight-chain or branched C₁ to C₆ alkyl groups or cyclic C₃ to C₆ alkyl groups, and
R³ is H.

It is particularly preferable when
R¹ is H, C₁ to C₆ alkyl, where C₁ to C₆ alkyl comprises straight-chain or branched C₁ to C₆ alkyl groups or cyclic C₃ to C₆ alkyl groups, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R² is H or methyl, and
R³ is H.

Compounds of the general formula (I) are in equilibrium with their keto form.

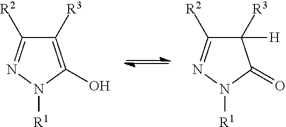

In their enol form, compounds of the general formula (I) can undergo addition reactions with (meth)acrylic acid and/or C₁ to C₈ alkyl (meth)acrylates. In mixtures of compounds of the general formula (I) with (meth)acrylic acid and/or C₁ to C₈ alkyl (meth)acrylates, the corresponding addition products may accordingly be present. Where R³ is H, in addition to single addition, double addition can also occur.

The addition of (meth)acrylic acid to 5-methyl-2-phenyl-4H-pyrazol-3-one is illustrated by way of example for the addition of (meth)acrylic acid to compounds of the general formula (I).

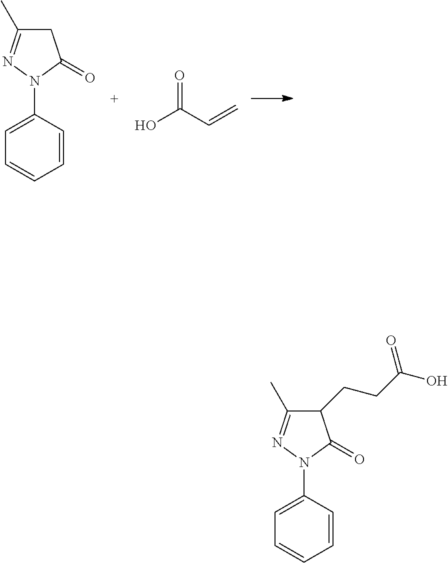

In accordance with the addition of (meth)acrylic acid to 5-methyl-2-phenyl-4H-pyrazol-3-one, (meth)acrylic esters can likewise undergo addition to compounds of the general formula (I). In addition to single addition, double addition is also possible.

Likewise disclosed is a mixture comprising one or more compounds of the general formula (I) and (meth)acrylic acid and/or C₁ to C₈ alkyl (meth)acrylates.

In compounds of the general formula (II),

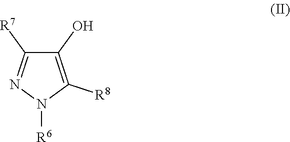

R⁶ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
R⁷ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl, and
R⁸ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl.

In more preferred compounds of the general formula (II),
R⁶ is H or C₁ to C₆ alkyl, where C₁ to C₆ alkyl comprises straight-chain or branched C₁ to C₆ alkyl groups or cyclic C₃ to C₆ alkyl groups,
R⁷ is H, C₁ to C₆ alkyl, where C₁ to C₆ alkyl comprises straight-chain or branched C₁ to C₆ alkyl groups or cyclic C₃ to C₆ alkyl groups, alkanoyl having C₁ to C₆ alkyl, where C₁ to C₆ alkyl comprises straight-chain or branched C₁ to C₆ alkyl groups or cyclic C₃ to C₆ alkyl groups, and
R⁸ is H or C₁ to C₆ alkyl, where C₁ to C₆ alkyl comprises straight-chain or branched C₁ to C₆ alkyl groups or cyclic C₃ to C₆ alkyl groups.

Likewise disclosed is a mixture comprising one or more compounds of the general formula (II) and (meth)acrylic acid and/or C₁ to C₈ alkyl (meth)acrylates.

In compounds of the general formula (III),

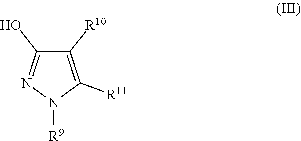

R⁹ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R¹⁰ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl, and
R¹¹ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl.

Likewise disclosed is a mixture comprising one or more compounds of the general formula (III) and (meth)acrylic acid and/or C₁ to C₈ alkyl (meth)acrylates.

In compounds of the general formula (IV),

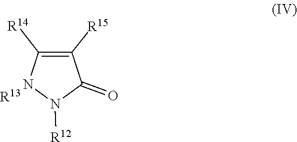

R¹² is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R¹³ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R¹⁴ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl, and
R¹⁵ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, 4-isopropoxyphenyl, NH₂, NH(CO)R¹⁶ where R¹⁶ is H, C₁ to C₆ alkyl, where C₁ to C₆ alkyl comprises straight-chain or branched C₁ to C₆ alkyl groups or cyclic C₃ to C₆ alkyl groups, phenyl, tolyl such as m- or p-tolyl, 4-methoxyphenyl or dimethylphenyl, such as 3,4-dimethylphenyl.

Likewise disclosed is a mixture comprising one or more compounds of the general formula (IV) and (meth)acrylic acid and/or C₁ to C₈ alkyl (meth)acrylates.

C₁ to C₈ alkyl (meth)acrylates are preferably methyl (meth)acrylate, ethyl (meth)acrylate, n-butyl (meth)acrylate, 2-ethylhexyl (meth)acrylate or a mixture of two or more of said (meth)acrylic esters.

The preparation of compounds of the general formula (I), (II), (III) or (IV) is known to those skilled in the art or established by them based on their general knowledge of the art.

The total amount of compounds of the general formula (I), (II), (III) or (IV) in the mixture is preferably 0.1 to 1000 ppm by weight based on the total weight of the amount of (meth)acrylic acid and C₁ to C₈ alkyl (meth)acrylates present in the mixture. The total amount of compounds of the general formula (I), (II), (III) or (IV) in the mixture is particularly preferably 1 to 900 ppm by weight, very particularly preferably 10 to 800 ppm by weight, and in particular 10 to 500 ppm by weight, in each case based on the total weight of the amount of (meth)acrylic acid and C₁ to C₈ alkyl (meth)acrylates present in the mixture.

If, in addition to (meth)acrylic acid and/or C₁ to C₈ alkyl (meth)acrylates and one or more compounds of the general formula (I), (II), (III) or (IV), the mixture also comprises other components, the total amount of (meth)acrylic acid and C₁ to C₈ alkyl (meth)acrylates in the mixture according to the invention is preferably at least 5% by weight based on the total weight of the mixture, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight.

Examples of other components are solvents, such as organic solvents, water or solvent mixtures, dissolved gases such as air, secondary components, co-stabilizers or any desired mixtures of a plurality of these components.

Examples of secondary components are by-products formed during the production and/or purification of (meth)acrylic acid and/or C₁ to C₈ alkyl (meth)acrylates and incompletely removed during the purification thereof. Secondary components may also be various contaminants introduced during production, purification, and/or storage of (meth)acrylic acid and/or C₁ to C₈ alkyl (meth)acrylates. Secondary components may be introduced into the mixture in the same way via the compounds of the general formula (I), (II), (III) or (IV).

(Meth)acrylic acid may, depending on production and purification, contain one or more secondary components. Examples of secondary components that may be present are water, di(meth)acrylic acid, acetic acid, propionic acid, one or more aldehydes, such as acetaldehyde, acrolein, propionaldehyde, 2-furfural, 3-furfural, coumarone or benzaldehyde, protoanemonin, maleic acid, maleic anhydride, allyl acrylate or any desired mixture of two or more of said secondary components. The content of secondary components is generally within a range from the determinable limit of detection to 20 000 ppm by weight based on the amount of (meth)acrylic acid.

C₁ to C₈ alkyl (meth)acrylates may, depending on their production and purification, contain one or more secondary components. Examples of secondary components that may be present are water, (meth)acrylic acid, alcohols of the respective (meth)acrylic esters, propionic esters with the alcohols of the respective (meth)acrylic esters, acetic esters with the alcohols of the respective (meth)acrylic esters, ethers of the respective (meth)acrylic ester alcohols or any desired mixture of two or more of said secondary components. The content of secondary components is generally within a range from the determinable limit of detection to 20 000 ppm by weight based on the amount of (meth)acrylic esters.

The mixture according to the invention is preferably stored and/or transported under an oxygen-containing atmosphere. The oxygen-containing atmosphere preferably has an oxygen content of 5 to 30 vol % of oxygen, in particular an oxygen content of 5 to 21 vol %.

In order for oxygen saturation of the mixture according to the invention to be achieved as rapidly as possible and/or for continuous oxygen saturation to be ensured, it may be preferable to introduce an oxygen-containing gas into the mixture. The oxygen-containing gas may be introduced into the mixture for example via one or more nozzles positioned such that the openings thereof extend into the mixture.

The oxygen-containing gas is for example air, nitrogen-air mixtures or oxygen-nitrogen mixtures other than air having an oxygen content of 5 to 30 vol %. In addition to oxygen and nitrogen, oxygen-nitrogen mixtures may contain other gases such as carbon dioxide, carbon monoxide, hydrogen, methane, one or more noble gases or a mixture of two or more of said gases.

The mixture may be produced by mixing one or more compounds of the general formula (I), (II), (III) or (IV) with (meth)acrylic acid and/or C₁ to C₈ alkyl (meth)acrylates. It may be expedient to produce a concentrate of compounds of the general formula (I), (II), (III) or (IV) in (meth)acrylic acid, C₁ to C₈ alkyl (meth)acrylates, and/or in a suitable solvent and to then dilute the concentrate thus obtained with (meth)acrylic acid and/or C₁ to C₈ alkyl (meth)acrylates. When choosing a suitable solvent, which may also be a solvent mixture, those skilled in the art are guided by general, expedient considerations such as solubility, miscibility, and secondary effects that could potentially occur. (Meth)acrylic acid and/or C₁ to C₈ alkyl (meth)acrylates may be added to the concentrate, or the concentrate may be added to (meth)acrylic acid and/or C₁ to C₈ alkyl (meth)acrylates.

The mixture preferably comprises a co-stabilizer. A co-stabilizer may be present in the mixture according to the invention as individual chemical compounds or as a mixture of compounds. A co-stabilizer helps prevent or retard the free-radical polymerization of (meth)acrylic acid and/or C₁ to C₈ alkyl (meth)acrylates. The costabilizer may act synergistically with the compounds of the general formula (I), (II), (III) or (IV) in preventing or retarding the free-radical polymerization of (meth)acrylic acid and/or C₁ to C₈ alkyl (meth)acrylates. The co-stabilizer may be added to the mixture before and/or during storage and/or transport. The co-stabilizer may be added to the (meth)acrylic acid and/or C₁ to C₈ alkyl (meth)acrylates together with or separately from the compounds of the general formula (I), (II), (III) or (IV). The co-stabilizer may however already have been added during production and/or purification of the (meth)acrylic acid and/or C₁ to C₈ alkyl (meth)acrylates, in which case compounds of the general formula (I), (II), (III) or (IV) are added subsequently to the present mixture.

The total amount of co-stabilizer in the mixtures according to the invention is preferably 0.1 to 5000 ppm by weight based on the total amount of (meth)acrylic acid and C₁ to C₈ alkyl (meth)acrylates in the mixture. The total amount of co-stabilizer is more preferably 1 to 4000 ppm by weight, in particular 5 to 2500 ppm by weight, and particularly preferably 50 to 750 ppm by weight, in each case based on the total amount of (meth)acrylic acid and C₁ to C₈ alkyl (meth)acrylates in the mixture.

The amount of co-stabilizer may be adjusted through addition or removal. Methods for removing the co-stabilizer are known to those skilled in the art (see e.g. U.S. Pat. No. 6,046,357). Examples of suitable methods include distillation, crystallization, ion exchange, extraction, reverse osmosis, and/or membrane methods. When choosing a suitable method or combination of methods, those skilled in the art are guided by their general knowledge of the art. If compounds of the general formula (I), (II), (III) or (IV) are removed with them, these may be optionally replenished. If the content of the co-stabilizer in the mixture is reduced or eliminated, it may also optionally be necessary to increase the content of compounds of the general formula (I), (II), (III) or (IV) in order to achieve the desired stabilization of the polymerization.

Examples of co-stabilizers are metal salts consisting of the group of copper, manganese, and cerium salts, phenothiazines, phenolic compounds, N-oxyls, phenylenediamines, nitroso compounds, ureas, thioureas, or mixtures of two or more of said substance classes. The co-stabilizer preferably is phenothiazines, phenolic compounds, N-oxyls or mixtures of two or more of said substance classes.

Copper salts are preferably copper(I) and/or copper(II) salts having sufficient solubility in the polymerizable compounds and in the mixture according to the invention. Examples of suitable copper salts are copper(II) phenolate, copper(II) acetylacetonate, copper(II) gluconate, copper(II) tartrate, copper(II) acetate, copper(II) formate, copper(II) nitrate, copper(II) hydroxide, copper(II) sulfate, copper(II) carbonate, copper(II) naphthenate, copper(II) acrylate, copper(II) halides such as copper(II) chloride, copper(II) salicylate, copper(II) sulfonate, copper(II) propionate, copper(II) octanate, copper(I) chloride, copper(I) iodide, copper(I) bromide, copper(I) cyanide, copper(I) acetate, copper(I) oxide, copper(I) sulfate, complex copper salts such as Cu(NH₃)₄⁺ with suitable counterions, ligand-stabilized copper complexes such as those disclosed in DE 10 2012 223 695 A1, or any desired mixtures of two or more of said copper salts. The copper salts concerned may also be used in the form of the hydrates thereof.

Manganese salts are preferably manganese(II) salts having sufficient solubility in the polymerizable compounds and in the mixture according to the invention. Examples of suitable manganese salts are manganese(II) phenolate, manganese(II) acetylacetonate, manganese(II) gluconate, manganese(II) tartrate, manganese(II) acetate, manganese(II) formate, manganese(II) nitrate, manganese(II) hydroxide, manganese(II) sulfate, manganese(II) carbonate, manganese(II) naphthenate, manganese(II) acrylate, manganese(II) halides such as manganese(II) chloride, manganese(II) salicylate, manganese(II) sulfonate, manganese(II) propionate, manganese(II) octanate, ligand-stabilized manganese complexes such as those disclosed in DE 10 2012 223 695 A1, or any desired mixtures of two or more of said manganese salts. The manganese salts concerned may also be used in the form of the hydrates thereof.

Cerium salts are preferably cerium(III) salts having sufficient solubility in the polymerizable compounds and in the mixture according to the invention. Examples of suitable cerium salts are cerium(III) phenolate, cerium(II) acetylacetonate, cerium(III) gluconate, cerium(II) tartrate, cerium(II) acetate, cerium(II) formate, cerium(III) nitrate, cerium(II) hydroxide, cerium(II) sulfate, cerium(III) carbonate, cerium(III) naphthenate, cerium(II) acrylate, cerium(III) halides such as cerium(III) chloride, cerium(III) salicylate, cerium(II) sulfonate, cerium(III) propionate, cerium(III) octanate, ligand-stabilized cerium complexes such as those disclosed in DE 10 2012 223 695 A1, or any desired mixtures of two or more of said cerium salts. The cerium salts concerned may also be used in the form of the hydrates thereof.

Examples of phenothiazines are bis(α-methylbenzyl)phenothiazine, 3,7-dioctylphenothiazine, bis(α-dimethylbenzyl)phenothiazine or any desired mixture of two or more of said compounds.

Examples of phenolic compounds are hydroquinone, hydroquinone monomethyl ether, such as para-methoxyphenol (MEHQ), pyrogallol, catechol, resorcinol, phenol, cresol, 2,4-dimethyl-6-tert-butylphenol, 2,6-di-tert-butyl-para-cresol or any desired mixture of two or more of said compounds.

Examples of N-oxyls are di-tert-butyl nitroxide, 2,2,6,6-tetramethyl-4-hydroxypiperidyl-1-oxyl, 2,2,6,6-tetramethylpiperidyl-1-oxyl, 2,2,6,6-tetramethylpiperidinoxyl, 4-hydroxy-2,2,6,6-tetramethylpiperidinoxyl, 4,4′,4″-tris-1-(2,2,6,6-tetramethylpiperidinoxyl)phosphites or any desired mixture of two or more of said compounds.

The present application further provides for the use of one or more compounds of the general formula (I), (II), (III) or (IV) as storage and/or transport stabilizers for (meth)acrylic acid and/or C₁ to C₈ alkyl (meth)acrylates. Reference is made in full to the preferred embodiments already described. Embodiments that are particularly preferred, very particularly preferred or characterized as preferred through similar formulations are thus also covered.

The present application additionally provides a method for stabilizing (meth)acrylic acid and/or C₁ to C₈ alkyl (meth)acrylates during transport and/or storage through the use of one or more compounds of the general formula (I), (II), (III) or (IV). Reference is made in full to the preferred embodiments already described. Embodiments that are particularly preferred, very particularly preferred or characterized as preferred through similar formulations are thus also covered.

The method according to the invention comprises the provision of a mixture comprising one or more compounds of the general formula (I), (II), (III) or (IV) and (meth)acrylic acid and/or C₁ to C₈ alkyl (meth)acrylates, the mixture being 50 to 100%, preferably 90 to 100%, and more preferably 95 to 100%, saturated with oxygen. The degree of oxygen saturation refers to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.

The present invention also provides a method for polymerizing (meth)acrylic acid and/or C₁ to C₈ alkyl (meth)acrylates in the presence of one or more compounds of the general formula (I), (II), (III) or (IV), the reaction mixture being 0 to 50%, preferably 0 to 10%, more preferably 0 to 5%, particularly preferably 0 to 1%, most preferably 0 to 0.1%, saturated with oxygen. The degree of oxygen saturation refers to the degree of oxygen saturation that is achieved when the mixture is stored and/or transported under standard conditions under an oxygen-containing atmosphere having an oxygen content of 5 to 30 vol % and preferably having an oxygen content of 5 to 21 vol %.

If the oxygen saturation of the reaction mixture is too high, the oxygen saturation may be reduced, for example by introducing nitrogen into the reaction mixture and/or into the atmosphere above the reaction mixture. The nitrogen introduced should be largely free of oxygen, for example an oxygen content of less than 1 vol %, preferably less than 0.1 vol %.

In addition to (meth)acrylic acid and/or C₁ to C₈ alkyl (meth)acrylates, the reaction mixture may also comprise other polymerizable compounds.

Reference is made in full to the preferred embodiments already described. Embodiments that are particularly preferred, very particularly preferred or characterized as preferred through similar formulations are thus also covered.

It is preferable that the polymerization is carried out at a temperature of 50 to 150° C. and more preferably at a temperature of 70 to 120° C.

It is preferable that the polymerization is carried out under a nitrogen atmosphere. A nitrogen atmosphere has a nitrogen content of 85 to 100 vol %, preferably 90 to 100 vol %, and more preferably 99 to 100%. The oxygen content of the nitrogen atmosphere is below 5 vol %.

It is preferable that the polymerization is carried out in the presence of a polymerization initiator. Suitable as polymerization initiators are the polymerization initiators known to those skilled in the art, such as those described for example in The Chemistry of Radical Polymerisation, 2nd edition, 2005, pages 49 to 166. The polymerization initiator is used in effective amounts.

Through the method according to the invention it is possible for (meth)acrylic acid and/or C₁ to C₈ alkyl (meth)acrylates that are storage and/or transport-stabilized with one or more compounds of the general formula (I), (II), (III) or (IV) to undergo polymerization without needing to first separate the compounds of the general formula (I), (II), (III) or (IV) from the reaction mixture to be polymerized. The reaction mixture may thus be initially charged in stabilized form in the reactor used for the polymerization. Through lowering the oxygen saturation in the reaction mixture, preferably by introducing nitrogen into the space in the reactor above the reaction mixture and/or by introducing nitrogen into the reaction mixture, the stabilizing effect of the compounds of the general formula (I), (II), (III) or (IV) can be reduced to a degree sufficient for polymerization of the reaction mixture to occur. The polymerization initiator may be added before, during or after the lowering of the oxygen saturation. For safety reasons, it is preferable when the polymerization initiator is added before lowering the oxygen saturation.

The present invention covers in particular the following embodiments.
1. A mixture comprising one or more compounds of the general formula (II)

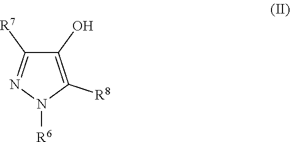

where
R⁶ is H, C₁ to C₂₀ alkyl, where C₁ to C₂₀ alkyl comprises straight-chain or branched C₁ to C₂₀ alkyl groups or cyclic C₃ to C₂₀ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
R⁷ is H, C₁ to C₂₀ alkyl, where C₁ to C₂₀ alkyl comprises straight-chain or branched C₁ to C₂₀ alkyl groups or cyclic C₃ to C₂₀ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl, and
R⁸ is H, C₁ to C₂₀ alkyl, where C₁ to C₂₀ alkyl comprises straight-chain or branched C₁ to C₂₀ alkyl groups or cyclic C₃ to C₂₀ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
and (meth)acrylic acid and/or C₁ to C₈ alkyl (meth)acrylates.
2. The mixture according to embodiment 1, wherein
   R⁶ is H, C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
   R⁷ is H, C₁ to C₈ alkyl, where C₁ to C₈ alkyl comprises straight-chain or branched C₁ to C₈ alkyl groups or cyclic C₃ to C₈ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl, and
   R⁸ is H, C₁ to C₈ alkyl, where C₁ to C₈ alkyl comprises straight-chain or branched C₁ to C₈ alkyl groups or cyclic C₃ to C₈ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl.
3. The mixture according to embodiment 1, wherein
   R⁶ is H or C₁ to C₆ alkyl, where C₁ to C₆ alkyl comprises straight-chain or branched C₁ to C₆ alkyl groups or cyclic C₃ to C₆ alkyl groups,
   R⁷ is H, C₁ to C₆ alkyl, where C₁ to C₆ alkyl comprises straight-chain or branched C₁ to C₆ alkyl groups or cyclic C₃ to C₆ alkyl groups, or alkanoyl having C₁ to C₆ alkyl, where C₁ to C₆ alkyl comprises straight-chain or branched C₁ to C₆ alkyl groups or cyclic C₃ to C₆ alkyl groups, and
   R⁸ is H or C₁ to C₆ alkyl, where C₁ to C₆ alkyl comprises straight-chain or branched C₁ to C₆ alkyl groups or cyclic C₃ to C₆ alkyl groups.
4. The mixture according to any of embodiments 1 to 3, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
5. The mixture according to any of embodiments 1 to 4, wherein the total amount of compounds of the general formula (II) in the mixture is 0.1 to 1000 ppm by weight, preferably 1 to 900 ppm by weight, more preferably 10 to 800 ppm by weight, and in particular 10 to 500 ppm by weight, in each case based on the total weight of the amount of (meth)acrylic acid and C₁ to C₈ alkyl (meth)acrylates present in the mixture.
6. The mixture according to any of embodiments 1 to 5, wherein the total amount of (meth)acrylic acid and C₁ to C₈ alkyl (meth)acrylates in the mixture is at least 5% by weight, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight, in each case based on the total weight of the mixture.
7. The mixture according to any of embodiments 1 to 3 or 5 to 6, wherein the mixture comprises (meth)acrylic acid and wherein the mixture comprises one or more of the following components:
   a. water 20 to 15 000 ppm by weight based on the total weight of (meth)acrylic acid present in the mixture,
   b. di(meth)acrylic acid 100 to 20 000 ppm by weight based on the total weight of (meth)acrylic acid present in the mixture,
   c. acetic acid 100 to 2000 ppm by weight based on the total weight of (meth)acrylic acid present in the mixture,
   d. propionic acid 100 to 2000 ppm by weight based on the total weight of (meth)acrylic acid present in the mixture,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the total weight of (meth)acrylic acid present in the mixture,
   f. protoanemonin 0 to 2 ppm by weight based on the total weight of (meth)acrylic acid present in the mixture,
   g. maleic acid 0 to 1 ppm by weight based on the total weight of (meth)acrylic acid present in the mixture,
   h. maleic anhydride 0 to 1 ppm by weight based on the total weight of (meth)acrylic acid present in the mixture,
   i. allyl acrylate 0 to 30 ppm by weight, preferably 0 to 1 ppm by weight, based on the total weight of (meth)acrylic acid present in the mixture.
8. The mixture according to any of embodiments 4 to 6, wherein the mixture comprises one or more (meth)acrylic esters according to embodiment 4 and wherein the mixture comprises one or more of the following components:
   a. water 1 to 2000 ppm by weight, preferably 1 to 500 ppm by weight, in each case based on the total weight of (meth)acrylic esters according to embodiment 4 present in the mixture,
   b. (meth)acrylic acid 1 to 2000 ppm by weight, preferably 1 to 100 ppm by weight, in each case based on the total weight of (meth)acrylic esters according to embodiment 4 present in the mixture,
   c. alcohols of the respective (meth)acrylic esters 0.01 to 2000 ppm by weight, preferably 0.1 to 1000 ppm by weight, in each case based on the total weight of (meth)acrylic esters according to embodiment 4 present in the mixture,
   d. propionic esters with the alcohols of the respective (meth)acrylic esters 0.1 to 2000 ppm by weight, preferably 10 to 1000 ppm by weight, in each case based on the total weight of (meth)acrylic esters according to embodiment 4 present in the mixture,
   e. acetic esters with the alcohols of the respective (meth)acrylic esters 1 to 2000 ppm by weight, preferably 5 to 2000 ppm by weight, in each case based on the total weight of (meth)acrylic esters according to embodiment 4 present in the mixture,
   f. ethers of the respective (meth)acrylic ester alcohols 0 to 2000 ppm by weight, in each case based on the total weight of (meth)acrylic esters according to embodiment 4 present in the mixture,
9. The mixture according to any of embodiments 1 to 8, wherein the mixture is stored and/or transported under an oxygen-containing atmosphere.
10. The mixture according embodiment 9, wherein the oxygen-containing atmosphere has an oxygen content of 5 to 30 vol % oxygen and preferably an oxygen content of 5 to 21 vol %.
11. The mixture according embodiment 9 or 10, wherein the mixture is stored and/or transported under an oxygen-containing atmosphere for 10 seconds or longer, preferably for 1 minute or longer, more preferably for 10 minutes or longer, and particularly preferably for 60 minutes or longer.
12. The mixture according to any of embodiments 1 to 11, wherein the mixture comprises a co-stabilizer.
13. The mixture according to embodiment 12, wherein the total amount of co-stabilizer present in the mixture is 0.1 to 5000 ppm by weight, preferably 1 to 4000 ppm by weight, in particular 5 to 2500 ppm by weight, and more preferably 50 to 750 ppm by weight, based on the total weight of the amount of (meth)acrylic acid and C₁ to C₈ alkyl (meth)acrylates present in the mixture.
14. The mixture according either of embodiments 12 or 13, wherein the co-stabilizer is metal salts from the group consisting of copper, manganese, and cerium salts, phenothiazines, phenolic compounds, N-oxyls, phenylenediamines, nitroso compounds, ureas, thioureas, or mixtures of two or more of said substance classes.
15. The use of one or more compounds of the general formula (II) as defined in any of embodiments 1 to 3 for the storage- and/or transport-stabilization of (meth)acrylic acid and/or C₁ to C₈ alkyl (meth)acrylates.
16. The use according to embodiment 15, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
17. The use according to either of embodiments 15 or 16, wherein the compounds of the general formula (II) are used in a total amount of 0.1 to 1000 ppm by weight, preferably 1 to 900 ppm by weight, more preferably 10 to 800 ppm by weight, and in particular 10 to 500 ppm by weight, based on the total weight of the amount of (meth)acrylic acid and C₁ to C₈ alkyl (meth)acrylates to be stabilized.
18. The use according to either of embodiments 15 or 17, wherein (meth)acrylic acid is stabilized and wherein the (meth)acrylic acid to be stabilized comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, in each case based on the amount of (meth)acrylic acid.
19. The use according to either of embodiments 16 or 17, wherein one or more (meth)acrylic esters according to embodiment 16 are stabilized and the (meth)acrylic esters to be stabilized comprise one or more of the following components:
   a. water 1 to 2000 ppm by weight, preferably 1 to 500 ppm by weight, in each case based on the amount of (meth)acrylic esters according to embodiment 16,
   b. (meth)acrylic acid 1 to 2000 ppm by weight, preferably 1 to 100 ppm by weight, in each case based on the amount of (meth)acrylic esters according to embodiment 16,
   c. alcohols of the respective (meth)acrylic esters 0.01 to 2000 ppm by weight, preferably 0.1 to 1000 ppm by weight, in each case based on the amount of (meth)acrylic esters according to embodiment 16,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 16,
   e. acetic esters with the alcohols of the respective (meth)acrylic esters 1 to 2000 ppm by weight, preferably 5 to 2000 ppm by weight, in each case based on the amount of (meth)acrylic esters according to embodiment 16,
   f. ethers of the respective (meth)acrylic ester alcohols 0 to 2000 ppm by weight based on the amount of (meth)acrylic esters according to embodiment 16,
20. A method for polymerizing (meth)acrylic acid and/or C₁ to C₈ alkyl (meth)acrylates in the presence of one or more compounds of the general formula (II) as defined in any of embodiments 1 to 3, the reaction mixture being 0 to 50%, preferably 0 to 10%, more preferably 0 to 5%, particularly preferably 0 to 1%, most preferably 0 to 0.1%, saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.
21. The method according to embodiment 20, wherein the total amount of compounds of the general formula (II) in the reaction mixture is 0.1 to 1000 ppm by weight, preferably 1 to 900 ppm by weight, more preferably 10 to 800 ppm by weight, and in particular 10 to 500 ppm by weight, in each case based on the total weight of the amount of (meth)acrylic acid and C₁ to C₈ alkyl (meth)acrylates present in the reaction mixtures.
22. The method according to either of embodiments 20 or 21, wherein the polymerization is carried out at a temperature of 50 to 150° C. and preferably 70 to 120° C.
23. The method according to any of embodiments 20 to 22, wherein the polymerization is carried out under a nitrogen atmosphere having a nitrogen content of 85 to 100 vol % and an oxygen content of less than 5 vol %, preferably a nitrogen content of 90 to 100 vol %, and more preferably a nitrogen content of 99 to 100 vol %.
24. The method according to any of embodiments 20 to 23, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
25. The method according to any of embodiments 20 or 23, wherein the (meth)acrylic acid to be polymerized comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, based on the amount of (meth)acrylic acid.
26. The method according to embodiment 24, wherein the (meth)acrylic esters to be polymerized comprise one or more of the following components:
   a. water 1 to 2000 ppm by weight, preferably 1 to 500 ppm by weight, in each case based on the amount of (meth)acrylic esters according to embodiment 24,
   b. (meth)acrylic acid 1 to 2000 ppm by weight, preferably 1 to 100 ppm by weight, in each case based on the amount of (meth)acrylic esters according to embodiment 24,
   c. alcohols of the respective (meth)acrylic esters 0.01 to 2000 ppm by weight, preferably 0.1 to 1000 ppm by weight, in each case based on the amount of (meth)acrylic esters according to embodiment 24,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 24,
   e. acetic esters with the alcohols of the respective (meth)acrylic esters 1 to 2000 ppm by weight, preferably 5 to 2000 ppm by weight, in each case based on the amount of (meth)acrylic esters according to embodiment 24,
   f. ethers of the respective (meth)acrylic ester alcohols 0 to 2000 ppm by weight based on the amount of (meth)acrylic esters according to embodiment 24,
27. A method for stabilizing (meth)acrylic acid and/or C₁ to C₈ alkyl (meth)acrylates through the use of one or more compounds of the general formula (II) as defined in any of embodiments 1 to 3.
28. The method according to embodiment 27, wherein (meth)acrylic acid and/or C₁ to C₈ alkyl (meth)acrylates are stabilized during storage and/or transport.
29. The method according to either of embodiments 27 or 28, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
30. The method according to either of embodiments 27 or 28, wherein the (meth)acrylic acid comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, based on the amount of (meth)acrylic acid.
31. The method according to embodiment 29, wherein the (meth)acrylic esters comprise one or more of the following components:
   a. water 1 to 2000 ppm by weight, preferably 1 to 500 ppm by weight, in each case based on the amount of (meth)acrylic esters according to embodiment 29,
   b. (meth)acrylic acid 1 to 2000 ppm by weight, preferably 1 to 100 ppm by weight, in each case based on the amount of (meth)acrylic esters according to embodiment 29,
   c. alcohols of the respective (meth)acrylic esters 0.01 to 2000 ppm by weight, preferably 0.1 to 1000 ppm by weight, in each case based on the amount of (meth)acrylic esters according to embodiment 29,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 29,
   e. acetic esters with the alcohols of the respective (meth)acrylic esters 1 to 2000 ppm by weight, preferably 5 to 2000 ppm by weight, in each case based on the amount of (meth)acrylic esters according to embodiment 29,
   f. ethers of the respective (meth)acrylic ester alcohols 0 to 2000 ppm by weight based on the amount of (meth)acrylic esters according to embodiment 29,
32. The method according to any of embodiments 27 to 31, wherein the method comprises the provision of a mixture that comprises one or more compounds of the general formula (II) and (meth)acrylic acid and/or C₁ to C₈ alkyl (meth)acrylates, the mixture being 50 to 100%, preferably 90 to 100%, and more preferably 95 to 100%, saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.
33. The method according to embodiment 32, wherein the total amount of (meth)acrylic acid and C₁ to C₈ alkyl (meth)acrylates in the mixture is at least 5% by weight based on the total weight of the mixture, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight.
34. The method according to any of embodiments 27 to 33, wherein the compounds of the general formula (II) are used in a total amount of 0.1 to 1000 ppm by weight, preferably 1 to 900 ppm by weight, more preferably 10 to 800 ppm by weight, and in particular 10 to 500 ppm by weight, in each case based on the total weight of the (meth)acrylic acid and C₁ to C₈ alkyl (meth)acrylates present in the mixture.
35. A mixture comprising one or more compounds of the general formula (I)

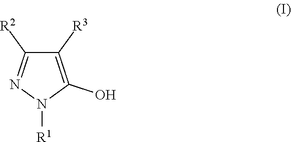

where
R¹ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R² is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl, and
R³ is H, C₂ to C₂₀ alkyl, where C₂ to C₂₀ alkyl comprises straight-chain or branched C₂ to C₂₀ alkyl groups or cyclic C₃ to C₂₀ alkyl groups, preferred alkyl radicals being C₁ to C₀ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl, and
and (meth)acrylic acid and/or C₁ to C₈ alkyl (meth)acrylates.
36. The mixture according to embodiment 35, wherein R¹ is H, C₁ to C₆ alkyl, where C₁ to C₆ alkyl comprises straight-chain or branched C₁ to C₆ alkyl groups or cyclic C₃ to C₆ alkyl groups, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
   R² is —H or C₁ to C₆ alkyl, and
   R³ is H.
37. The mixture according to embodiment 35, wherein
   R¹ is H, C₁ to C₆ alkyl, where C₁ to C₆ alkyl comprises straight-chain or branched C₁ to C₆ alkyl groups or cyclic C₃ to C₆ alkyl groups, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
   R² is H or methyl, and
   R³ is H.
38. The mixture according to any of embodiments 35 to 37, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
39. The mixture according to any of embodiments 35 to 38, wherein the total amount of compounds of the general formula (I) in the mixture is 0.1 to 1000 ppm by weight, preferably 1 to 900 ppm by weight, more preferably 10 to 800 ppm by weight, and in particular 10 to 500 ppm by weight, in each case based on the total weight of the amount of (meth)acrylic acid and C₁ to C₈ alkyl (meth)acrylates present in the mixture.
40. The mixture according to any of embodiments 35 to 39, wherein the total amount of (meth)acrylic acid and C₁ to C₈ alkyl (meth)acrylates in the mixture is at least 5% by weight, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight, in each case based on the total weight of the mixture.
41. The mixture according to any of embodiments 35 to 37 or 39 to 40, wherein the mixture comprises (meth)acrylic acid and wherein the mixture comprises one or more of the following components:
   a. water 20 to 15 000 ppm by weight based on the total weight of (meth)acrylic acid present in the mixture,
   b. di(meth)acrylic acid 100 to 20 000 ppm by weight based on the total weight of (meth)acrylic acid present in the mixture,
   c. acetic acid 100 to 2000 ppm by weight based on the total weight of (meth)acrylic acid present in the mixture,
   d. propionic acid 100 to 2000 ppm by weight based on the total weight of (meth)acrylic acid present in the mixture,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the total weight of (meth)acrylic acid present in the mixture,
   f. protoanemonin 0 to 2 ppm by weight based on the total weight of (meth)acrylic acid present in the mixture,
   g. maleic acid 0 to 1 ppm by weight based on the total weight of (meth)acrylic acid present in the mixture,
   h. maleic anhydride 0 to 1 ppm by weight based on the total weight of (meth)acrylic acid present in the mixture,
   i. allyl acrylate 0 to 30 ppm by weight, preferably 0 to 1 ppm by weight, based on the total weight of (meth)acrylic acid present in the mixture.
42. The mixture according to embodiment 38, wherein the mixture comprises one or more (meth)acrylic esters according to embodiment 38 and wherein the mixture comprises one or more of the following components:
   a. water 1 to 2000 ppm by weight, preferably 1 to 500 ppm by weight, in each case based on the total weight of (meth)acrylic esters according to embodiment 38 present in the mixture,
   b. (meth)acrylic acid 1 to 2000 ppm by weight, preferably 1 to 100 ppm by weight, in each case based on the total weight of (meth)acrylic esters according to embodiment 38 present in the mixture,
   c. alcohols of the respective (meth)acrylic esters 0.01 to 2000 ppm by weight, preferably 0.1 to 1000 ppm by weight, in each case based on the total weight of (meth)acrylic esters according to embodiment 38 present in the mixture,
   d. propionic esters with the alcohols of the respective (meth)acrylic esters 0.1 to 2000 ppm by weight, preferably 10 to 1000 ppm by weight, in each case based on the total weight of (meth)acrylic esters according to embodiment 38 present in the mixture,
   e. acetic esters with the alcohols of the respective (meth)acrylic esters 1 to 2000 ppm by weight, preferably 5 to 2000 ppm by weight, in each case based on the total weight of (meth)acrylic esters according to embodiment 38 present in the mixture,
   f. ethers of the respective (meth)acrylic ester alcohols 0 to 2000 ppm by weight, in each case based on the total weight of (meth)acrylic esters according to embodiment 38 present in the mixture,
43. The mixture according to any of embodiments 35 to 42, wherein the mixture is stored and/or transported under an oxygen-containing atmosphere.
44. The mixture according to embodiment 43, wherein the oxygen-containing atmosphere has an oxygen content of 5 to 30 vol % oxygen and preferably an oxygen content of 5 to 21 vol %.
45. The mixture according to embodiment 43 or 44, wherein the mixture is stored and/or transported under an oxygen-containing atmosphere for 10 seconds or longer, preferably for 1 minute or longer, more preferably for 10 minutes or longer, and particularly preferably for 60 minutes or longer.
46. The mixture according to any of embodiments 35 to 45, wherein the mixture comprises a co-stabilizer.
47. The mixture according to embodiment 46, wherein the total amount of co-stabilizer present in the mixture is 0.1 to 5000 ppm by weight, preferably 1 to 4000 ppm by weight, in particular 5 to 2500 ppm by weight, and more preferably 50 to 750 ppm by weight, based on the total weight of the amount of (meth)acrylic acid and C₁ to C₈ alkyl (meth)acrylates present in the mixture.
48. The mixture according either of embodiments 46 or 47, wherein the co-stabilizer is metal salts from the group consisting of copper, manganese, and cerium salts, phenothiazines, phenolic compounds, N-oxyls, phenylenediamines, nitroso compounds, ureas, thioureas, or mixtures of two or more of said substance classes.
49. The use of one or more compounds of the general formula (I) as defined in any of embodiments 35 to 37 for the storage- and/or transport-stabilization of (meth)acrylic acid and/or C₁ to C₈ alkyl (meth)acrylates.
50. The use according to embodiment 49, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
51. The use according to either of embodiments 49 or 50, wherein the compounds of the general formula (I) are used in a total amount of 0.1 to 1000 ppm by weight, preferably 1 to 900 ppm by weight, more preferably 10 to 800 ppm by weight, and in particular 10 to 500 ppm by weight, based on the total weight of the amount of (meth)acrylic acid and C₁ to C₈ alkyl (meth)acrylates to be stabilized.
52. The use according to either of embodiments 49 or 51, wherein (meth)acrylic acid is stabilized and wherein the (meth)acrylic acid to be stabilized comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, in each case based on the amount of (meth)acrylic acid.
53. The use according to embodiment 50 or 51, wherein one or more (meth)acrylic esters according to embodiment 50 is stabilized and the (meth)acrylic esters to be stabilized comprise one or more of the following components:
   a. water 1 to 2000 ppm by weight, preferably 1 to 500 ppm by weight, in each case based on the amount of (meth)acrylic esters according to embodiment 50,
   b. (meth)acrylic acid 1 to 2000 ppm by weight, preferably 1 to 100 ppm by weight, in each case based on the amount of (meth)acrylic esters according to embodiment 50,
   c. alcohols of the respective (meth)acrylic esters 0.01 to 2000 ppm by weight, preferably 0.1 to 1000 ppm by weight, in each case based on the amount of (meth)acrylic esters according to embodiment 50,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 50,
   e. acetic esters with the alcohols of the respective (meth)acrylic esters 1 to 2000 ppm by weight, preferably 5 to 2000 ppm by weight, in each case based on the amount of (meth)acrylic esters according to embodiment 50,
   f. ethers of the respective (meth)acrylic ester alcohols 0 to 2000 ppm by weight based on the amount of (meth)acrylic esters according to embodiment 50.
54. A method for polymerizing (meth)acrylic acid and/or C₁ to C₈ alkyl (meth)acrylates in the presence of one or more compounds of the general formula (I) as defined in any of embodiments 35 to 37, the reaction mixture being 0 to 50%, preferably 0 to 10%, more preferably 0 to 5%, particularly preferably 0 to 1%, most preferably 0 to 0.1%, saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.
55. The method according to embodiment 54, wherein the total amount of compounds of the general formula (I) in the reaction mixture is 0.1 to 1000 ppm by weight, preferably 1 to 900 ppm by weight, more preferably 10 to 800 ppm by weight, and in particular 10 to 500 ppm by weight, in each case based on the total weight of the amount of (meth)acrylic acid and C₁ to C₈ alkyl (meth)acrylates present in the reaction mixtures.
56. The method according to either of embodiments 54 or 55, wherein the polymerization is carried out at a temperature of 50 to 150° C. and preferably 70 to 120° C.
57. The method according to any of embodiments 54 to 56, wherein the polymerization is carried out under a nitrogen atmosphere having a nitrogen content of 85 to 100 vol % and an oxygen content of less than 5 vol %, preferably a nitrogen content of 90 to 100 vol %, and more preferably a nitrogen content of 99 to 100%.
58. The method according to any of embodiments 54 to 58, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
59. The method according to any of embodiments 54 or 57, wherein the (meth)acrylic acid to be polymerized comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, based on the amount of (meth)acrylic acid.
60. The method according to embodiment 58, wherein the (meth)acrylic esters to be polymerized comprise one or more of the following components:
   a. water 1 to 2000 ppm by weight, preferably 1 to 500 ppm by weight, in each case based on the amount of (meth)acrylic esters according to embodiment 58,
   b. (meth)acrylic acid 1 to 2000 ppm by weight, preferably 1 to 100 ppm by weight, in each case based on the amount of (meth)acrylic esters according to embodiment 58,
   c. alcohols of the respective (meth)acrylic esters 0.01 to 2000 ppm by weight, preferably 0.1 to 1000 ppm by weight, in each case based on the amount of (meth)acrylic esters according to embodiment 58,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 58,
   e. acetic esters with the alcohols of the respective (meth)acrylic esters 1 to 2000 ppm by weight, preferably 5 to 2000 ppm by weight, in each case based on the amount of (meth)acrylic esters according to embodiment 58,
   f. ethers of the respective (meth)acrylic ester alcohols 0 to 2000 ppm by weight based on the amount of (meth)acrylic esters according to embodiment 58,
61. A method for stabilizing (meth)acrylic acid and/or C₁ to C₈ alkyl (meth)acrylates through the use of one or more compounds of the general formula (I) as defined in any of embodiments 35 to 37.
62. The method according to embodiment 61, wherein (meth)acrylic acid and/or C₁ to C₈ alkyl (meth)acrylates are stabilized during storage and/or transport.
63. The method according to either of embodiments 61 or 62, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
64. The method according to either of embodiments 61 or 62, wherein the (meth)acrylic acid comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, based on the amount of (meth)acrylic acid.
65. The method according to embodiment 63, wherein the (meth)acrylic esters comprise one or more of the following components:
   a. water 1 to 2000 ppm by weight, preferably 1 to 500 ppm by weight, in each case based on the amount of (meth)acrylic esters according to embodiment 63,
   b. (meth)acrylic acid 1 to 2000 ppm by weight, preferably 1 to 100 ppm by weight, in each case based on the amount of (meth)acrylic esters according to embodiment 63,
   c. alcohols of the respective (meth)acrylic esters 0.01 to 2000 ppm by weight, preferably 0.1 to 1000 ppm by weight, in each case based on the amount of (meth)acrylic esters according to embodiment 63,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 63,
   e. acetic esters with the alcohols of the respective (meth)acrylic esters 1 to 2000 ppm by weight, preferably 5 to 2000 ppm by weight, in each case based on the amount of (meth)acrylic esters according to embodiment 63,
   f. ethers of the respective (meth)acrylic ester alcohols 0 to 2000 ppm by weight based on the amount of (meth)acrylic esters according to embodiment 63,
66. The method according to any of embodiments 61 to 65, wherein the method comprises the provision of a mixture that comprises one or more compounds of the general formula (I) and (meth)acrylic acid and/or C₁ to C₈ alkyl (meth)acrylates, the mixture being 50 to 100%, preferably 90 to 100%, and more preferably 95 to 100%, saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.
67. The method according to embodiment 66, wherein the total amount of (meth)acrylic acid and C₁ to C₈ alkyl (meth)acrylates in the mixture is at least 5% by weight based on the total weight of the mixture, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight.
68. The method according to any of embodiments 61 to 67, wherein the compounds of the general formula (I) are used in a total amount of 0.1 to 1000 ppm by weight, preferably 1 to 900 ppm by weight, more preferably 10 to 800 ppm by weight, and in particular 10 to 500 ppm by weight, in each case based on the total weight of the (meth)acrylic acid and C₁ to C₈ alkyl (meth)acrylates present in the mixture.
69. A mixture comprising one or more compounds of the general formula (III)

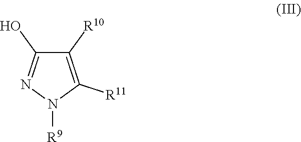

where
R⁹ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R¹⁰ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or is 4-isopropoxyphenyl, and
R¹¹ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or is 4-isopropoxyphenyl,
and (meth)acrylic acid and/or C₁ to C₈ alkyl (meth)acrylates.
70. The mixture according to embodiment 69, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
71. The mixture according to either of embodiments 69 or 70, wherein the total amount of compounds of the general formula (III) in the mixture is 0.1 to 1000 ppm by weight, preferably 1 to 900 ppm by weight, more preferably 10 to 800 ppm by weight, and in particular 10 to 500 ppm by weight, in each case based on the total weight of the amount of (meth)acrylic acid and C₁ to C₈ alkyl (meth)acrylates present in the mixture.
72. The mixture according to any of embodiments 69 to 71, wherein the total amount of (meth)acrylic acid and C₁ to C₈ alkyl (meth)acrylates in the mixture is at least 5% by weight, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight, in each case based on the total weight of the mixture.
73. The mixture according to any of embodiments 69, 71 or 72, wherein the mixture comprises (meth)acrylic acid and wherein the mixture comprises one or more of the following components:
   a. water 20 to 15 000 ppm by weight based on the total weight of (meth)acrylic acid present in the mixture,
   b. di(meth)acrylic acid 100 to 20 000 ppm by weight based on the total weight of (meth)acrylic acid present in the mixture,
   c. acetic acid 100 to 2000 ppm by weight based on the total weight of (meth)acrylic acid present in the mixture,
   d. propionic acid 100 to 2000 ppm by weight based on the total weight of (meth)acrylic acid present in the mixture,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the total weight of (meth)acrylic acid present in the mixture,
   f. protoanemonin 0 to 2 ppm by weight based on the total weight of (meth)acrylic acid present in the mixture,
   g. maleic acid 0 to 1 ppm by weight based on the total weight of (meth)acrylic acid present in the mixture,
   h. maleic anhydride 0 to 1 ppm by weight based on the total weight of (meth)acrylic acid present in the mixture,
   i. allyl acrylate 0 to 30 ppm by weight, preferably 0 to 1 ppm by weight, based on the total weight of (meth)acrylic acid present in the mixture.
74. The mixture according to any of embodiments 70 to 72, wherein the mixture comprises one or more (meth)acrylic esters according to embodiment 70 and wherein the mixture comprises one or more of the following components:
   a. water 1 to 2000 ppm by weight, preferably 1 to 500 ppm by weight, in each case based on the total weight of (meth)acrylic esters according to embodiment 70 present in the mixture,
   b. (meth)acrylic acid 1 to 2000 ppm by weight, preferably 1 to 100 ppm by weight, in each case based on the total weight of (meth)acrylic esters according to embodiment 70 present in the mixture,
   c. alcohols of the respective (meth)acrylic esters 0.01 to 2000 ppm by weight, preferably 0.1 to 1000 ppm by weight, in each case based on the total weight of (meth)acrylic esters according to embodiment 70 present in the mixture,
   d. propionic esters with the alcohols of the respective (meth)acrylic esters 0.1 to 2000 ppm by weight, preferably 10 to 1000 ppm by weight, in each case based on the total weight of (meth)acrylic esters according to embodiment 70 present in the mixture,
   e. acetic esters with the alcohols of the respective (meth)acrylic esters 1 to 2000 ppm by weight, preferably 5 to 2000 ppm by weight, in each case based on the total weight of (meth)acrylic esters according to embodiment 70 present in the mixture,
   f. ethers of the respective (meth)acrylic ester alcohols 0 to 2000 ppm by weight, in each case based on the total weight of (meth)acrylic esters according to embodiment 70 present in the mixture,
75. The mixture according to any of embodiments 69 to 74, wherein the mixture is stored and/or transported under an oxygen-containing atmosphere.
76. The mixture according to embodiment 75, wherein the oxygen-containing atmosphere has an oxygen content of 5 to 30 vol % oxygen and preferably an oxygen content of 5 to 21 vol %.
77. The mixture according to embodiment 75 or 76, wherein the mixture is stored and/or transported under an oxygen-containing atmosphere for 10 seconds or longer, preferably for 1 minute or longer, more preferably for 10 minutes or longer, and particularly preferably for 60 minutes or longer.
78. The mixture according to any of embodiments 69 to 77, wherein the mixture comprises a co-stabilizer.
79. The mixture according to embodiment 78, wherein the total amount of co-stabilizer present in the mixture is 0.1 to 5000 ppm by weight, preferably 1 to 4000 ppm by weight, in particular 5 to 2500 ppm by weight, and more preferably 50 to 750 ppm by weight, based on the total weight of the amount of (meth)acrylic acid and C₁ to C₈ alkyl (meth)acrylates present in the mixture.
80. The mixture according to either of embodiments 78 or 79, wherein the co-stabilizer is metal salts from the group consisting of copper, manganese, and cerium salts, phenothiazines, phenolic compounds, N-oxyls, phenylenediamines, nitroso compounds, ureas, thioureas, or mixtures of two or more of said substance classes.
81. The use of one or more compounds of the general formula (III) as defined in embodiment 69 for the storage- and/or transport-stabilization of (meth)acrylic acid and/or C₁ to C₈ alkyl (meth)acrylates.
82. The use according to embodiment 81, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
83. The use according to either of embodiments 81 or 82, wherein the compounds of the general formula (III) are used in a total amount of 0.1 to 1000 ppm by weight, preferably 1 to 900 ppm by weight, more preferably 10 to 800 ppm by weight, and in particular 10 to 500 ppm by weight, based on the total weight of the amount of (meth)acrylic acid and C₁ to C₈ alkyl (meth)acrylates to be stabilized.
84. The use according to either of embodiments 81 or 83, wherein (meth)acrylic acid is stabilized and wherein the (meth)acrylic acid to be stabilized comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, in each case based on the amount of (meth)acrylic acid.
85. The use according to either of embodiments 82 or 83, wherein one or more (meth)acrylic esters according to embodiment 82 are stabilized and the (meth)acrylic esters to be stabilized comprise one or more of the following components:
   a. water 1 to 2000 ppm by weight, preferably 1 to 500 ppm by weight, in each case based on the amount of (meth)acrylic esters according to embodiment 82,
b. (meth)acrylic acid 1 to 2000 ppm by weight, preferably 1 to 100 ppm by weight, in each case based on the amount of (meth)acrylic esters according to embodiment 82,
c. alcohols of the respective (meth)acrylic esters 0.01 to 2000 ppm by weight, preferably 0.1 to 1000 ppm by weight, in each case based on the amount of (meth)acrylic esters according to embodiment 82,
d. propionic esters with the alcohols of the respective (meth)acrylic esters 0.1 to 2000 ppm by weight, preferably 10 to 1000 ppm by weight, in each case based on the amount of (meth)acrylic esters according to embodiment 82,
   e. acetic esters with the alcohols of the respective (meth)acrylic esters 1 to 2000 ppm by weight, preferably 5 to 2000 ppm by weight, in each case based on the amount of (meth)acrylic esters according to embodiment 82,
   f. ethers of the respective (meth)acrylic ester alcohols 0 to 2000 ppm by weight based on the amount of (meth)acrylic esters according to embodiment 82.
86. A method for polymerizing (meth)acrylic acid and/or C₁ to C₈ alkyl (meth)acrylates in the presence of one or more compounds of the general formula (III) as defined in embodiment 69, the reaction mixture being 0 to 50%, preferably 0 to 10%, more preferably 0 to 5%, particularly preferably 0 to 1%, most preferably 0 to 0.1%, saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.
87. The method according to embodiment 86, wherein the total amount of compounds of the general formula (III) in the reaction mixture is 0.1 to 1000 ppm by weight, preferably 1 to 900 ppm by weight, more preferably 10 to 800 ppm by weight, and in particular 10 to 500 ppm by weight, in each case based on the total weight of the amount of (meth)acrylic acid and C₁ to C₈ alkyl (meth)acrylates present in the reaction mixtures.
88. The method according to either of embodiments 86 or 87, wherein the polymerization is carried out at a temperature of 50 to 150° C. and preferably 70 to 120° C.
89. The method according to any of embodiments 86 to 88, wherein the polymerization is carried out under a nitrogen atmosphere having a nitrogen content of 85 to 100 vol % and an oxygen content of less than 5 vol %, preferably a nitrogen content of 90 to 100 vol %, and more preferably a nitrogen content of 99 to 100%.
90. The method according to any of embodiments 86 to 89, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
91. The method according to any of embodiments 86 or 89, wherein the (meth)acrylic acid to be polymerized comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, based on the amount of (meth)acrylic acid.
92. The method according to embodiment 90, wherein the (meth)acrylic esters to be polymerized comprise one or more of the following components:
   a. water 1 to 2000 ppm by weight, preferably 1 to 500 ppm by weight, in each case based on the amount of (meth)acrylic esters according to embodiment 90,
   b. (meth)acrylic acid 1 to 2000 ppm by weight, preferably 1 to 100 ppm by weight, in each case based on the amount of (meth)acrylic esters according to embodiment 90,
   c. alcohols of the respective (meth)acrylic esters 0.01 to 2000 ppm by weight, preferably 0.1 to 1000 ppm by weight, in each case based on the amount of (meth)acrylic esters according to embodiment 90,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 90,
   e. acetic esters with the alcohols of the respective (meth)acrylic esters 1 to 2000 ppm by weight, preferably 5 to 2000 ppm by weight, in each case based on the amount of (meth)acrylic esters according to embodiment 90,
   f. ethers of the respective (meth)acrylic ester alcohols 0 to 2000 ppm by weight based on the amount of (meth)acrylic esters according to embodiment 90.
93. A method for stabilizing (meth)acrylic acid and/or C₁ to C₈ alkyl (meth)acrylates through the use of one or more compounds of the general formula (III) as defined in embodiment 69.
94. The method according to embodiment 93, wherein (meth)acrylic acid and/or C₁ to C₈ alkyl (meth)acrylates are stabilized during storage and/or transport.
95. The method according to either of embodiments 93 or 94, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
96. The method according to either of embodiments 93 or 94, wherein the (meth)acrylic acid comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, based on the amount of (meth)acrylic acid.
97. The method according to embodiment 95, wherein the (meth)acrylic esters comprise one or more of the following components:
   a. water 1 to 2000 ppm by weight, preferably 1 to 500 ppm by weight, in each case based on the amount of (meth)acrylic esters according to embodiment 95,
   b. (meth)acrylic acid 1 to 2000 ppm by weight, preferably 1 to 100 ppm by weight, in each case based on the amount of (meth)acrylic esters according to embodiment 95,
   c. alcohols of the respective (meth)acrylic esters 0.01 to 2000 ppm by weight, preferably 0.1 to 1000 ppm by weight, in each case based on the amount of (meth)acrylic esters according to embodiment 95,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 95,
   e. acetic esters with the alcohols of the respective (meth)acrylic esters 1 to 2000 ppm by weight, preferably 5 to 2000 ppm by weight, in each case based on the amount of (meth)acrylic esters according to embodiment 95,
   f. ethers of the respective (meth)acrylic ester alcohols 0 to 2000 ppm by weight based on the amount of (meth)acrylic esters according to embodiment 95,
98. The method according to any of embodiments 93 to 97, wherein the method comprises the provision of a mixture that comprises one or more compounds of the general formula (III) and (meth)acrylic acid and/or C₁ to C₈ alkyl (meth)acrylates, the mixture being 50 to 100%, preferably 90 to 100%, and more preferably 95 to 100%, saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.
99. The method according to embodiment 98, wherein the total amount of (meth)acrylic acid and C₁ to C₈ alkyl (meth)acrylates in the mixture is at least 5% by weight based on the total weight of the mixture, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight.
100. The method according to any of embodiments 93 to 99, wherein the compounds of the general formula (III) are used in a total amount of 0.1 to 1000 ppm by weight, preferably 1 to 900 ppm by weight, more preferably 10 to 800 ppm by weight, and in particular 10 to 500 ppm by weight, in each case based on the total weight of the (meth)acrylic acid and C₁ to C₈ alkyl (meth)acrylates present in the mixture.
101. A mixture comprising one or more compounds of the general formula (IV)

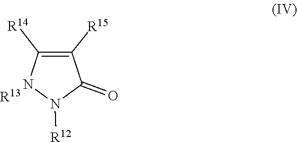

where
R¹² is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R¹³ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl,
R¹⁴ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, or 4-isopropoxyphenyl, and
R¹⁵ is H, C₁ to C₂₀ alkyl, where C₁ to C₂₀ alkyl comprises straight-chain or branched C₁ to C₂₀ alkyl groups or cyclic C₃ to C₂₀ alkyl groups, preferred alkyl radicals being C₁ to C₈ alkyl and more preferred alkyl radicals being C₁ to C₆ alkyl, phenyl, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl, carboxylic esters having C₁ to C₈ alkyl such as —COOMe, alkanoyl having C₁ to C₆ alkyl, where C₁ to C₆ alkyl comprises straight-chain or branched C₁ to C₆ alkyl groups or cyclic C₃ to C₆ alkyl groups, 4-isopropoxyphenyl, NH₂, NH(CO)R¹⁶ where R¹⁶ is H, C₁ to C₆ alkyl, phenyl, tolyl such as m- or p-tolyl, 4-methoxyphenyl or dimethylphenyl, such as 3,4-dimethylphenyl.
and (meth)acrylic acid and/or C₁ to C₈ alkyl (meth)acrylates.
102. The mixture according to embodiment 101, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
103. The mixture according to either of embodiments 101 or 102, wherein the total amount of compounds of the general formula (IV) in the mixture is 0.1 to 1000 ppm by weight, preferably 1 to 900 ppm by weight, more preferably 10 to 800 ppm by weight, and in particular 10 to 500 ppm by weight, in each case based on the total weight of the amount of (meth)acrylic acid and C₁ to C₈ alkyl (meth)acrylates present in the mixture.
104. The mixture according to any of embodiments 101 to 103, wherein the total amount of (meth)acrylic acid and C₁ to C₈ alkyl (meth)acrylates in the mixture is at least 5% by weight, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight, in each case based on the total weight of the mixture.
105. The mixture according to any of embodiments 101, 103 or 104, wherein the mixture comprises (meth)acrylic acid and wherein the mixture comprises one or more of the following components:
   a. water 20 to 15 000 ppm by weight based on the total weight of (meth)acrylic acid present in the mixture,
   b. di(meth)acrylic acid 100 to 20 000 ppm by weight based on the total weight of (meth)acrylic acid present in the mixture,
   c. acetic acid 100 to 2000 ppm by weight based on the total weight of (meth)acrylic acid present in the mixture,
   d. propionic acid 100 to 2000 ppm by weight based on the total weight of (meth)acrylic acid present in the mixture,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the total weight of (meth)acrylic acid present in the mixture,
   f. protoanemonin 0 to 2 ppm by weight based on the total weight of (meth)acrylic acid present in the mixture,
   g. maleic acid 0 to 1 ppm by weight based on the total weight of (meth)acrylic acid present in the mixture,
   h. maleic anhydride 0 to 1 ppm by weight based on the total weight of (meth)acrylic acid present in the mixture,
   i. allyl acrylate 0 to 30 ppm by weight, preferably 0 to 1 ppm by weight, based on the total weight of (meth)acrylic acid present in the mixture.
106. The mixture according to any of embodiments 102 to 104, wherein the mixture comprises one or more (meth)acrylic esters according to embodiment 102 and wherein the mixture comprises one or more of the following components:
   a. water 1 to 2000 ppm by weight, preferably 1 to 500 ppm by weight, in each case based on the total weight of (meth)acrylic esters according to embodiment 102 present in the mixture,
   b. (meth)acrylic acid 1 to 2000 ppm by weight, preferably 1 to 100 ppm by weight, in each case based on the total weight of (meth)acrylic esters according to embodiment 102 present in the mixture,
   c. alcohols of the respective (meth)acrylic esters 0.01 to 2000 ppm by weight, preferably 0.1 to 1000 ppm by weight, in each case based on the total weight of (meth)acrylic esters according to embodiment 102 present in the mixture,
   d. propionic esters with the alcohols of the respective (meth)acrylic esters 0.1 to 2000 ppm by weight, preferably 10 to 1000 ppm by weight, in each case based on the total weight of (meth)acrylic esters according to embodiment 102 present in the mixture,
   e. acetic esters with the alcohols of the respective (meth)acrylic esters 1 to 2000 ppm by weight, preferably 5 to 2000 ppm by weight, in each case based on the total weight of (meth)acrylic esters according to embodiment 102 present in the mixture,
   f. ethers of the respective (meth)acrylic ester alcohols 0 to 2000 ppm by weight, in each case based on the total weight of (meth)acrylic esters according to embodiment 102 present in the mixture.
107. The mixture according to any of embodiments 101 to 106, wherein the mixture is stored and/or transported under an oxygen-containing atmosphere.
108. The mixture according embodiment 107, wherein the oxygen-containing atmosphere has an oxygen content of 5 to 30 vol % oxygen and preferably an oxygen content of 5 to 21 vol %.
109. The mixture according embodiment 107 or 108, wherein the mixture is stored and/or transported under an oxygen-containing atmosphere for 10 seconds or longer, preferably for 1 minute or longer, more preferably for 10 minutes or longer, and particularly preferably for 60 minutes or longer.
110. The mixture according to any of embodiments 101 to 109, wherein the mixture comprises a co-stabilizer.
111. The mixture according to embodiment 110, wherein the total amount of costabilizer present in the mixture is 0.1 to 5000 ppm by weight, preferably 1 to 4000 ppm by weight, in particular 5 to 2500 ppm by weight, and more preferably 50 to 750 ppm by weight, based on the total weight of the amount of (meth)acrylic acid and C₁ to C₈ alkyl (meth)acrylates present in the mixture.
112. The mixture according either of embodiments 110 or 111, wherein the co-stabilizer is metal salts from the group consisting of copper, manganese, and cerium salts, phenothiazines, phenolic compounds, N-oxyls, phenylenediamines, nitroso compounds, ureas, thioureas, or mixtures of two or more of said substance classes.
113. The use of one or more compounds of the general formula (IV) as defined in embodiment 101 for the storage- and/or transport-stabilization of (meth)acrylic acid and/or C₁ to C₈ alkyl (meth)acrylates.
114. The use according to embodiment 113, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
115. The use according to either of embodiments 113 or 114, wherein the compounds of the general formula (IV) are used in a total amount of 0.1 to 1000 ppm by weight, preferably 1 to 900 ppm by weight, more preferably 10 to 800 ppm by weight, and in particular 10 to 500 ppm by weight, based on the total weight of the amount of (meth)acrylic acid and C₁ to C₈ alkyl (meth)acrylates to be stabilized.
116. The use according to either of embodiments 113 or 115, wherein (meth)acrylic acid is stabilized and wherein the (meth)acrylic acid to be stabilized comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, in each case based on the amount of (meth)acrylic acid.
117. The use according to either of embodiments 114 or 115, wherein one or more (meth)acrylic esters according to embodiment 114 are stabilized and the (meth)acrylic esters to be stabilized comprise one or more of the following components:
   a. water 1 to 2000 ppm by weight, preferably 1 to 500 ppm by weight, in each case based on the amount of (meth)acrylic esters according to embodiment 114,
   b. (meth)acrylic acid 1 to 2000 ppm by weight, preferably 1 to 100 ppm by weight, in each case based on the amount of (meth)acrylic esters according to embodiment 114,
   c. alcohols of the respective (meth)acrylic esters 0.01 to 2000 ppm by weight, preferably 0.1 to 1000 ppm by weight, in each case based on the amount of (meth)acrylic esters according to embodiment 114,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 114,
   e. acetic esters with the alcohols of the respective (meth)acrylic esters 1 to 2000 ppm by weight, preferably 5 to 2000 ppm by weight, in each case based on the amount of (meth)acrylic esters according to embodiment 114,
   f. ethers of the respective (meth)acrylic ester alcohols 0 to 2000 ppm by weight based on the amount of (meth)acrylic esters according to embodiment 114.
118. A method for polymerizing (meth)acrylic acid and/or C₁ to C₈ alkyl (meth)acrylates in the presence of one or more compounds of the general formula (IV) as defined in embodiment 101, the reaction mixture being 0 to 50%, preferably 0 to 10%, more preferably 0 to 5%, particularly preferably 0 to 1%, most preferably 0 to 0.1%, saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.
119. The method according to embodiment 118, wherein the total amount of compounds of the general formula (IV) in the reaction mixture is 0.1 to 1000 ppm by weight, preferably 1 to 900 ppm by weight, more preferably 10 to 800 ppm by weight, and in particular 10 to 500 ppm by weight, in each case based on the total weight of the amount of (meth)acrylic acid and C₁ to C₈ alkyl (meth)acrylates present in the reaction mixtures.
120. The method according to either of embodiments 118 or 119, wherein the polymerization is carried out at a temperature of 50 to 150° C. and preferably 70 to 120° C.
121. The method according to any of embodiments 118 to 120, wherein the polymerization is carried out under a nitrogen atmosphere having a nitrogen content of 85 to 100 vol % and an oxygen content of less than 5 vol %, preferably a nitrogen content of 90 to 100 vol %, and more preferably a nitrogen content of 99 to 100%.
122. The method according to any of embodiments 118 to 121, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
123. The method according to any of embodiments 118 to 121, wherein the (meth)acrylic acid to be polymerized comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, based on the amount of (meth)acrylic acid.
124. The method according to embodiment 122, wherein the (meth)acrylic esters to be polymerized comprise one or more of the following components:
   a. water 1 to 2000 ppm by weight, preferably 1 to 500 ppm by weight, in each case based on the amount of (meth)acrylic esters according to embodiment 122,
   b. (meth)acrylic acid 1 to 2000 ppm by weight, preferably 1 to 100 ppm by weight, in each case based on the amount of (meth)acrylic esters according to embodiment 122,
   c. alcohols of the respective (meth)acrylic esters 0.01 to 2000 ppm by weight, preferably 0.1 to 1000 ppm by weight, in each case based on the amount of (meth)acrylic esters according to embodiment 122,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 122,
   e. acetic esters with the alcohols of the respective (meth)acrylic esters 1 to 2000 ppm by weight, preferably 5 to 2000 ppm by weight, in each case based on the amount of (meth)acrylic esters according to embodiment 122,
   f. ethers of the respective (meth)acrylic ester alcohols 0 to 2000 ppm by weight based on the amount of (meth)acrylic esters according to embodiment 122.
125. A method for stabilizing (meth)acrylic acid and/or C₁ to C₈ alkyl (meth)acrylates through the use of one or more compounds of the general formula (IV) as defined in embodiment 101.
126. The method according to embodiment 125, wherein (meth)acrylic acid and/or C₁ to C₈ alkyl (meth)acrylates are stabilized during storage and/or transport.
127. The method according to either of embodiments 125 or 126, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.
128. The method according to either of embodiments 125 or 126, wherein the (meth)acrylic acid comprises one or more of the following components:
   a. water 20 to 15 000 ppm by weight based on the amount of (meth)acrylic acid,
   b. di(meth)acrylic acid 100 to 20 000 ppm by weight based on the amount of (meth)acrylic acid,
   c. acetic acid 100 to 2000 ppm by weight based on the amount of (meth)acrylic acid,
   d. propionic acid 100 to 2000 ppm by weight based on the amount of (meth)acrylic acid,
   e. one or more aldehydes, such as acetaldehyde, acrolein, propionaldehyde, 2-furfural, 3-furfural, coumarone or benzaldehyde, the amount of each aldehyde being 0 to 1 ppm by weight based on the amount of (meth)acrylic acid,
   f. protoanemonin 0 to 2 ppm by weight based on the amount of (meth)acrylic acid,
   g. maleic acid 0 to 1 ppm by weight based on the amount of (meth)acrylic acid,
   h. maleic anhydride 0 to 1 ppm by weight based on the amount of (meth)acrylic acid,
   i. allyl acrylate 0 to 30 ppm by weight, preferably 0 to 1 ppm by weight, based on the amount of (meth)acrylic acid.
129. The method according to embodiment 127, wherein the (meth)acrylic esters comprise one or more of the following components:
   a. water 1 to 2000 ppm by weight, preferably 1 to 500 ppm by weight, in each case based on the amount of (meth)acrylic esters according to embodiment 127,
   b. (meth)acrylic acid 1 to 2000 ppm by weight, preferably 1 to 100 ppm by weight, in each case based on the amount of (meth)acrylic esters according to embodiment 127,
   c. alcohols of the respective (meth)acrylic esters 0.01 to 2000 ppm by weight, preferably 0.1 to 1000 ppm by weight, in each case based on the amount of (meth)acrylic esters according to embodiment 127,
   d. propionic esters with the alcohols of the respective (meth)acrylic esters 0.1 to 2000 ppm by weight, preferably 10 to 1000 ppm by weight, in each case based on the amount of (meth)acrylic esters according to embodiment 127,
   e. acetic esters with the alcohols of the respective (meth)acrylic esters 1 to 2000 ppm by weight, preferably 5 to 2000 ppm by weight, in each case based on the amount of (meth)acrylic esters according to embodiment 127,
   f. ethers of the respective (meth)acrylic ester alcohols 0 to 2000 ppm by weight based on the amount of (meth)acrylic esters according to embodiment 127.
130. The method according to any of embodiments 125 to 129, wherein the method comprises the provision of a mixture that comprises one or more compounds of the general formula (IV) and (meth)acrylic acid and/or C₁ to C₈ alkyl (meth)acrylates, the mixture being 50 to 100%, preferably 90 to 100%, and more preferably 95 to 100%, saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol % and preferably having an oxygen content of 5 to 21 vol %.
131. The method according to embodiment 130, wherein the total amount of (meth)acrylic acid and C₁ to C₈ alkyl (meth)acrylates in the mixture is at least 5% by weight based on the total weight of the mixture, further preferably at least 20% by weight, further preferably at least 30% by weight, further preferably at least 40% by weight, further preferably at least 50% by weight, further preferably at least 60% by weight, further preferably at least 70% by weight, further preferably at least 80% by weight, further preferably at least 90% by weight, and particularly preferably at least 95% by weight.
132. The method according to any of embodiments 125 to 131, wherein the compounds of the general formula (IV) are used in a total amount of 0.1 to 1000 ppm by weight, preferably 1 to 900 ppm by weight, more preferably 10 to 800 ppm by weight, and in particular 10 to 500 ppm by weight, in each case based on the total weight of the (meth)acrylic acid and C₁ to C₈ alkyl (meth)acrylates present in the mixture.

### EXAMPLES

The examples that follow do not restrict the subject matter of the present invention.

| Starting material | Manufacturer | Purity |
|---|---|---|
| Acrylic acid | BASF SE | ≥99% |
| Toluene | BASF SE | >99.7% |
| 2,2′-Azobis(4-methoxy-2,4- | Fujifilm Wako Chemicals | n.a. |
| dimethylvaleronitrile) | Europe GmbH | |
| 4-Methoxyphenol | Sigma-Aldrich | ≥98% |
| Ethyl acrylate | BASF SE | ≥99.7% |

The acrylic acid used in the experiments was freshly distilled to remove the stabilizer contained therein. The distilled acrylic acid has a purity of greater than 99.5% by weight. Secondary components present in the distilled acrylic acid are acetic acid (0.0726% by weight), diacrylic acid (0.0497% by weight), and propionic acid (0.0184% by weight).

Weights reported in the experiment descriptions, such as percent by weight (% by weight) or ppm by weight values based on acrylic acid, are based on the mass of acrylic acid used in each case including the secondary components contained therein.

Experiments examining the inhibition of polymerization under air or nitrogen:

For the comparative experiments, an acrylic acid solution containing 40 ppm by weight of 4-methoxyphenol (MEHQ) was prepared. The stated ppm by weight value is based on the mass of the acrylic acid used.

For further comparative experiments, mixtures of acrylic acid with compounds different from the compounds of the general formula (I), (II), (III) or (IV) were prepared, the molar amounts of the compounds used corresponding to the molar amount of MEHQ in the comparative experiments.

For the inventive experiments, mixtures of acrylic acid with compounds of the general formula (I), (II), (III) and/or (IV) were prepared, the molar amounts of the compounds of the general formula (I), (II), (III) and/or (IV) in the mixtures corresponding to the molar amount of MEHQ in the comparative experiments.

Exemplary Dilution Series:

2.5 g of an acrylic acid solution having an MEHQ content of 1000 ppm by weight based on the mass of acrylic acid used was diluted with acrylic acid to an MEHQ content of 500 ppm by weight based on the mass of acrylic acid used.

2.5 g of the acrylic acid solution thus obtained was diluted with acrylic acid to an MEHQ content of 250 ppm by weight based on the mass of acrylic acid used.

2.5 g of the acrylic acid solution thus obtained was diluted with acrylic acid to an MEHQ content of 125 ppm by weight based on the mass of acrylic acid used.

2.5 g of the acrylic acid solution thus obtained was diluted with acrylic acid to an MEHQ content of 80 ppm by weight based on the mass of acrylic acid used.

2.5 g of the acrylic acid solution thus obtained was diluted with acrylic acid to an MEHQ content of 40 ppm by weight based on the mass of acrylic acid used.

The initiator solution used was a freshly prepared toluene solution of 2,2′-azobis(4-methoxy-2,4-dimethylvaleronitrile). The content of 2,2′-azobis(4-methoxy-2,4-dimethylvaleronitrile) in the initiator solution is 6420 ppm by weight based on the mass of toluene in the initiator solution.

Tests of the Inhibition of Polymerization Under Air:

2.2 g of acrylic acid solution containing 0.000715 mmol of the respective polymerization stabilizer was initially charged under air. To the initially charged acrylic acid solution was added 0.22 g of initiator solution and the mixture was homogenized. 1.9 ml of the mixture thus obtained was transferred in air to a 4.5 ml reaction vessel. The reaction vessel was closed airtight and fitted with a temperature sensor. The temperature sensor extended into the mixture in the reaction vessel.

The reaction vessel containing the mixture was then placed in a heating block heated to 40° C.

Once the mixture had reached a temperature of 40° C., the temperature profile over time was measured. The period from reaching 40° C. until reaching the maximum temperature is used as the reference value for the inhibition power of the respective polymerization inhibitor.

For a mixture with MEHQ as polymerization stabilizer, a rise in temperature of 145° C. to a maximum temperature of 185° C. was measured, said rise lasting 42 seconds. The maximum temperature was reached 9188 seconds after the mixture had thermally equilibrated at 40° C. In the calculation of the efficiency of the polymerization stabilizers relative to MEHQ, the value of 9188 seconds corresponds to an efficiency of 100 seconds.

For a mixture with 5-methyl-2-phenyl-4H-pyrazol-3-one as polymerization stabilizer, a rise in temperature of 125° C. to a maximum temperature of 165° C. was observed, said rise lasting 48 seconds. The maximum temperature was reached 7874 seconds after the mixture had thermally equilibrated at 40° C.

The efficiency of 5-methyl-2-phenyl-4H-pyrazol-3-one as a polymerization stabilizer was thus 86 percent relative to MEHQ.

Tests of the Inhibition of Polymerization Under Nitrogen:

2.2 g of acrylic acid solution containing 0.000715 mmol of the respective polymerization stabilizer was initially charged under air. To the initially charged acrylic acid solution was added 0.22 g of initiator solution and the mixture was homogenized. 1.9 ml of the mixture thus obtained was transferred in air to a 4.5 ml reaction vessel. The reaction vessel was closed airtight and fitted with a temperature sensor. The temperature sensor extended into the mixture in the reaction vessel. Nitrogen was introduced into the reaction vessel above the initially charged mixture, with equalization of pressure. The nitrogen flow rate was 3 to 4 I/h. The inflow time was 20 seconds. The nitrogen had a purity of greater than 99.9%.

The reaction vessel containing the mixture was placed in a heating block. The mixture was then heated in the heating block to 40° C. over a period of 4 minutes.

Once the mixture had reached a temperature of 40° C., the temperature profile over time was measured. The period from reaching 40° C. until reaching the maximum temperature is used as the reference value for the inhibition power of the respective polymerization inhibitor.

For a mixture with MEHQ as polymerization stabilizer, a rise in temperature of 133° C. to a maximum temperature of 173° C. was measured, said rise lasting 54 seconds. The maximum temperature was reached 3978 seconds after the mixture had thermally equilibrated at 40° C. In the calculation of the efficiency of the polymerization stabilizers relative to MEHQ, the value of 3978 seconds corresponds to an efficiency of 100%.

For a mixture with 5-methyl-2-phenyl-4H-pyrazol-3-one as polymerization stabilizer, a rise in temperature of 119° C. to a maximum temperature of 159° C. was observed, said rise lasting 66 seconds. The maximum temperature was reached 1056 seconds after the mixture had thermally equilibrated at 40° C.

The efficiency of 5-methyl-2-phenyl-4H-pyrazol-3-one as a polymerization stabilizer was thus 27 percent relative to MEHQ.

Results of the tests in acrylic acid with non-inventive compounds:

| | | Relative | Relative |
|---|---|---|---|
| | | stabilization of | stabilization |
| | | acrylic acid | of acrylic |
| | | under air | acid under |
| | | relative to | nitrogen relative |
| Structure | IUPAC name | MeHQ (100%) | to MeHQ (100%) |
| 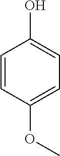 | 4-Methoxy- phenol | 100 | 100 |
| 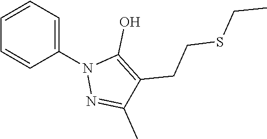 [SMILES: CCSCCc1c(C)nn(-c2ccccc2)c1O] | 4-(2-Ethyl- sulfanylethyl)-5- methyl-2-phenyl- pyrazol-3-ol | 71 | 64 |
| 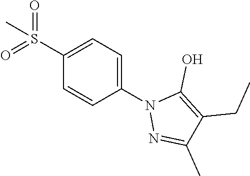 [SMILES: CCc1c(C)nn(-c2ccc(S(C)(=O)=O)cc2)c1O] | 4-(2-Ethyl- sulfanylethyl)- 5-methyl-2-(4- methylsulfonyl- phenyl)pyrazol- 3-ol | 68 | 50 |
| 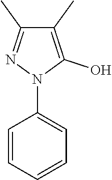 | 4,5-Dimethyl- 2-phenyl- pyrazol-3-ol | 57 | 71 |
| 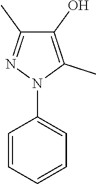 | 3,5-Dimethyl- 1-phenyl- pyrazol-4-ol | 134 | 154 |
| 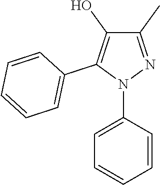 [SMILES: Cc1nn(-c2ccccc2)c(-c2ccccc2)c1O] | 3-Methyl- 1,5-diphenyl- pyrazol-4-ol | 97 | 161 |

Results of the Tests in Acrylic Acid with Inventive Compounds:

| | | Relative sta- | |
|---|---|---|---|
| | | bilization of | |
| | | acrylic acid | Relative stabili- |
| | | under air rel- | zation of acrylic |
| | | ative to | acid under nitro- |
| | | MeHQ | gen relative to |
| Structure | IUPAC name | (100%) | MeHQ (100%) |
| 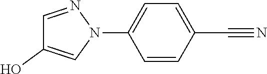 [SMILES: N#Cc1ccc(-n2cc(O)cn2)cc1] | 4-(4-Hydroxy- pyrazol-1- yl)benzonitrile | 85 | 33 |
| 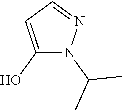 | 2-Isopropyl- pyrazol-3-ol | 83 | 72 |
| 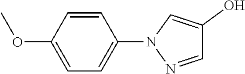 | 1-(4-Methoxy- phenyl) pyrazol-4-ol | 95 | 43 |
| 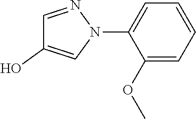 | 1-(2-Methoxy- phenyl) pyrazol-4-ol | 96 | 51 |
| 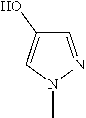 | 1-Methyl- pyrazol-4-ol | 93 | 53 |
| 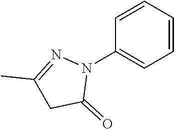 | 5-Methyl-2- phenyl-4H- pyrazol-3-one | 86 | 27 |
| 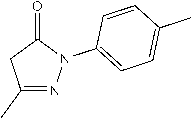 | 5-Methyl-2- (p-tolyl)-4H- pyrazol-3-one | 103 | 39 |
| 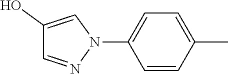 | 1-(p-Tolyl)- pyrazol-4-ol | 89 | 51 |
| 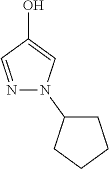 [SMILES: Oc1cnn(C2CCCC2)c1] | 1-Cyclopentyl- pyrazol-4-ol | 144 | 104 |
| 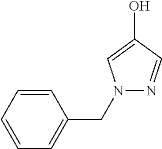 [SMILES: Oc1cnn(Cc2ccccc2)c1] | 1-Benzyl- pyrazol-4-ol | 91 | 46 |
| 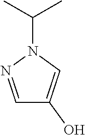 | 1-Isopropyl- pyrazol-4-ol | 89 | 80 |
| 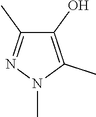 | 1,3,5-Trimethyl- pyrazol-4- ol | 102 | 87 |
| 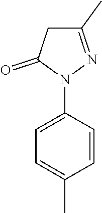 | 2-(4-Chloro- phenyl)-5- methyl-4H- pyrazol-3-one | 78 | 12 |
| 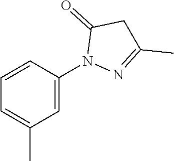 | 5-Methyl- 2-(m-tolyl)- 4H-pyrazol-3- one | 77 | 15 |
| 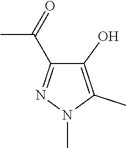 | 1-(4-Hydroxy- 1,5-dimethyl- pyrazol-3-yl)- ethanone | 97% | 34% |
| 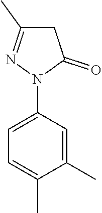 | 2-(3,4- Dimethylphenyl)-5- methyl-4H- pyrazol-3-one | 83 | 25 |
| 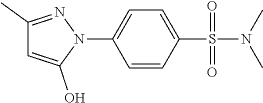 | 4-(5-Hydroxy- 3-methyl- pyrazol-1-yl)-N,N- dimethyl- benzene- sulfonamide | 75 | 13 |
| 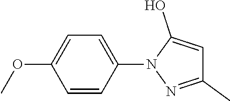 | 2-(4-Methoxy- phenyl)-5- methyl- pyrazol-3-ol | 84 | 16 |
| 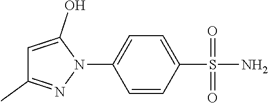 [SMILES: Cc1cc(O)n(-c2ccc(S(N)(=O)=O)cc2)n1] | 4-(5-Hydroxy- 3-methylpyrazol- 1-yl)benzene- sulfonamide | 78 | 11 |
| 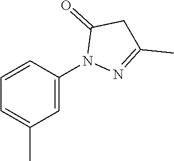 | 5-Methyl- 2-(m-tolyl)- 4H-pyrazol-3- one | 77 | 15 |
| 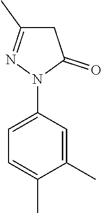 | 2-(3,4- Dimethylphenyl)-5- methyl-4H- pyrazol-3-one | 82 | 25 |
| 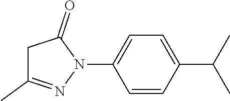 [SMILES: CC1=NN(c2ccc(C(C)C)cc2)C(=O)C1] | 2-(4-Isopropyl- phenyl)-5- methyl-4H- pyrazol-3-one | 81 | 24 |
| 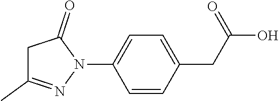 [SMILES: CC1=NN(c2ccc(CC(=O)O)cc2)C(=O)C1] | 2-[4-(3- Methyl-5-oxo-4H- pyrazol-1- yl)phenyl]- acetic acid | 82 | 20 |
| 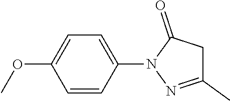 | 2-(4-Methoxy- phenyl)-5- methyl-4H- pyrazol-3-one | 75 | 13 |
| 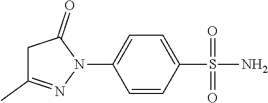 [SMILES: CC1=NN(c2ccc(S(N)(=O)=O)cc2)C(=O)C1] | 4-(3-Methyl-5- oxo-4H- pyrazol-1-yl)- benzene- sulfonamide | 78 | 11 |
| 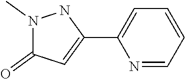 | 2-Methyl-5- (2-pyridyl)-1H- pyrazol-3-one | 79 | 18 |
| 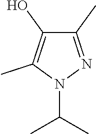 | 1-Isopropyl- 3,5-dimethyl- pyrazol-4-ol | 238 | 122 |

Polymerization Experiments:

A reaction vessel was charged under a nitrogen atmosphere with 450 g of deionized water. The initially charged reaction mixture was heated to 95° C. with stirring. On reaching a temperature of 95° C., three streams were fed in while maintaining the temperature.
Stream 1: 500 g of acrylic acid, fed in over 5 h
Stream 2: 15 g of sodium hypophosphite in 35 g of deionized water, fed in over 4.75 h
Stream 3: 5 g of sodium persulfate in 66.4 g of deionized water, fed in over 5.25 h

After addition of the three streams, the reaction mixture was stirred at 95° C. for a further hour. The reaction mixture was then cooled to room temperature and 80 g of deionized water added.

In the inventive experiment, the acrylic acid from stream 1 was stabilized with 280 ppm by weight of edaravone (5-methyl-2-phenyl-4H-pyrazol-3-one) based on the mass of the acrylic acid. In the comparative experiment, the acrylic acid from stream 1 was stabilized with 200 ppm by weight of MEHQ based on the mass of the acrylic acid.

The polymers obtained were analyzed by GPC (calibration with Na-PAA standard, eluent 0.01 mol/l phosphate buffer pH 7.4 in dist. water containing 0.01 M NaN₃).

| | Mw (g/mol) |
|---|---|
| Polymer (edaravone) | 9560 |
| Polymer (MEHQ) | 9400 |

Tests in Acrylates:

Experiments Examining the Inhibition of Polymerization Under Air or Nitrogen:

The experiments examining the inhibition of polymerization under air or nitrogen were carried out using ethyl acrylate with the stabilizer removed. The ethyl acrylate used for this, which contains 10 to 20 ppm by weight of MEHQ based on the amount of ethyl acrylate, was filtered through type 90 active neutral alumina without application of pressure. For each 100 ml of stabilized ethyl acrylate, 20 g of type 90 active neutral alumina was used. After the filtration, MEHQ was no longer detectable by HPLC analysis in the ethyl acrylate.

For the comparative experiments, an ethyl acrylate solution containing 13 ppm by weight of MEHQ was prepared. The ppm by weight value is based on the mass of the ethyl acrylate present in the solution.

For further comparative experiments, mixtures of ethyl acrylate with compounds different from the compounds of the general formula (I), (II), (III) or (IV) were prepared, the molar amounts of the compounds used corresponding to the molar amount of MEHQ in the comparative experiments.

For the inventive experiments, mixtures of ethyl acrylate with compounds of the general formula (I), (II), (III) or (IV) were prepared, the molar amounts of the compounds of the general formula (I), (II), (III) or (IV) in the mixtures corresponding to the molar amount of MEHQ in the comparative experiments.

Exemplary Dilution Series:

1.0 g of an ethyl acrylate solution having an MEHQ content of 500 ppm by weight based on the mass of the ethyl acrylate present in the solution was diluted with ethyl acrylate to an MEHQ content of 240 ppm by weight based on the mass of the ethyl acrylate in the solution.

1.5 g of the ethyl acrylate solution thus obtained was diluted with ethyl acrylate to an MEHQ content of 80 ppm by weight based on the mass of the ethyl acrylate in the solution.

1.5 g of the ethyl acrylate solution thus obtained was diluted with ethyl acrylate to an MEHQ content of 30 ppm by weight based on the mass of the ethyl acrylate in the solution.

2.0 g of the ethyl acrylate solution thus obtained was diluted with ethyl acrylate to an MEHQ content of 13 ppm by weight based on the mass of the ethyl acrylate in the solution.

The initiator solution used was a freshly prepared toluene solution of 2,2′-azobis(4-methoxy-2,4-dimethylvaleronitrile). The content of 2,2′-azobis(4-methoxy-2,4-dimethylvaleronitrile) in the initiator solution is 6420 ppm by weight based on the mass of toluene in the initiator solution.

Tests of the Inhibition of Polymerization Under Air:

2.0 g of ethyl acrylate solution containing 0.00021 mmol of the respective polymerization stabilizer was initially charged under air. To the initially charged ethyl acrylate solution was added 0.17 g of initiator solution and the mixture was homogenized. 1.9 ml of the mixture thus obtained was transferred in air to a 4.5 ml reaction vessel. The reaction vessel was closed airtight and fitted with a temperature sensor. The temperature sensor extended into the mixture in the reaction vessel.

The reaction vessel containing the mixture was placed in a heating block heated to 40° C.

Once the mixture had reached a temperature of 40° C., the temperature profile over time was measured. The period from reaching 40° C. until reaching the maximum temperature is used as the reference value for the inhibition power of the respective polymerization inhibitor.

For a mixture with MEHQ as polymerization stabilizer, a rise in temperature of 8° C. to a maximum temperature of 48° C. was measured, said rise lasting 1200 seconds. The maximum temperature was reached 29084 seconds after the mixture had thermally equilibrated at 40° C. In the calculation of the efficiency of the polymerization stabilizers relative to MEHQ, the value of 29084 seconds corresponds to an efficiency of 100%.

For an inventive mixture with 5-methyl-2-phenyl-4H-pyrazol-3-one as polymerization stabilizer, a rise in temperature of 6° C. to a maximum temperature of 46° C. was observed, said rise lasting 1200 seconds. The maximum temperature was reached 28470 seconds after the mixture had thermally equilibrated at 40° C. The efficiency of 5-methyl-2-phenyl-4H-pyrazol-3-one as a polymerization stabilizer was thus 98 percent relative to MEHQ.

Tests of the Inhibition of Polymerization Under Nitrogen:

2.0 g of ethyl acrylate solution containing 000021 mmol of the respective polymerization stabilizer was initially charged under air. To the initially charged ethyl acrylate solution was added 0.17 g of initiator solution and the mixture was homogenized. 1.9 ml of the mixture thus obtained was transferred in air to a 4.5 ml reaction vessel. The reaction vessel was closed airtight and fitted with a temperature sensor. The temperature sensor extended into the mixture in the reaction vessel. Nitrogen was introduced into the reaction vessel above the initially charged mixture, with equalization of pressure. The nitrogen flow rate was 3 to 4 I/h. The inflow time was 20 seconds. The nitrogen had a purity of greater than 99.9%.

The reaction vessel containing the mixture was placed in a heating block. The mixture was then heated in the heating block to 40° C. over a period of 4 minutes.

Once the mixture had reached a temperature of 40° C., the temperature profile over time was measured. The period from reaching 40° C. until reaching the maximum temperature is used as the reference value for the inhibition power of the respective polymerization inhibitor.

For a mixture with MEHQ as polymerization stabilizer, a rise in temperature of 73° C. to a maximum temperature of 113° C. was measured, said rise lasting 240 seconds. The maximum temperature was reached 460 seconds after the mixture had thermally equilibrated at 40° C. In the calculation of the efficiency of the polymerization stabilizers relative to MEHQ, the value of 460 seconds corresponds to an efficiency of 100 seconds.

For an inventive mixture with 5-methyl-2-phenyl-4H-pyrazol-3-one as polymerization stabilizer, a rise in temperature of 80° C. to a maximum temperature of 120° C. was observed, said rise lasting 240 seconds. The maximum temperature was reached 426 seconds after the mixture had thermally equilibrated at 40° C. The efficiency of 5-methyl-2-phenyl-4H-pyrazol-3-one as a polymerization stabilizer was thus 93 percent relative to MEHQ.

Results of the Tests in Ethyl Acrylate with Inventive Compounds:

| | | Relative | |
|---|---|---|---|
| | | stabilization of | Relative |
| | | ethyl | stabilization |
| | | acrylate under | of ethyl acrylate |
| | | air relative | under nitrogen |
| | | to MeHQ | relative to |
| Structure | IUPAC name | (100%) | MeHQ (100%) |
| 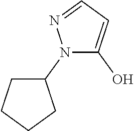 [SMILES: Oc1ccnn1C1CCCC1] | 2-Cyclopentyl- pyrazol-3-ol | 97 | 45 |
| 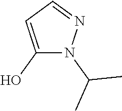 | 2-Isopropyl- pyrazol-3-ol | 95 | 69 |
| 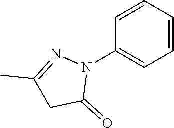 | 5-Methyl-2- phenyl-4H- pyrazol-3-one | 86 | 45 |
| 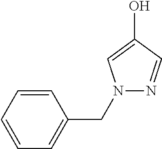 [SMILES: Oc1cnn(Cc2ccccc2)c1] | 1-Benzyl- pyrazol-4-ol | 107 | 58 |
| 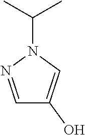 | 1-Isopropyl- pyrazol-4-ol | 106 | 101 |
| 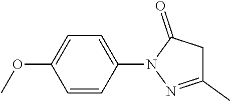 | 2-(4-Methoxy- phenyl)-5- methyl-4H-pyrazol- 3-one | 89 | 46 |
| 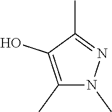 | 1,3,5-Trimethyl- pyrazol-4-ol | 117 | 97 |
| 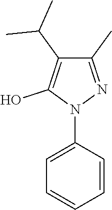 [SMILES: Cc1nn(-c2ccccc2)c(O)c1C(C)C] | 4-Isopropyl-5- methyl-2-phenyl- pyrazol-3-ol | 151 | 63 |
| 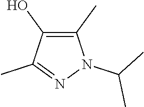 | 1-Isopropyl- 3,5-dimethyl- pyrazol-4-ol | 122 | 86 |

Polymerization Experiments with Acrylates:

Homopolymerization of n-Butyl Acrylate:

Comparative Experiment:

A reaction vessel was charged under a nitrogen atmosphere with 210 g of n-butyl acetate, 14.7 g of n-butyl acrylate (stabilized with 15 ppm by weight of MEHQ based on the amount of n-butyl acrylate), and 0.08 g of azobisisobutyronitrile (AIBN). The initially charged reaction mixture was heated to 80° C. with stirring. 15 minutes after reaching a temperature of 80° C., two streams were fed in while maintaining the temperature.
Stream 1: 195.3 g of n-butyl acrylate (stabilized with 15 ppm by weight of MEHQ based on the amount of n-butyl acrylate) and 100.5 g of n-butyl acetate, fed in over 2 h
Stream 2: 0.98 g of AIBN and 74.1 g of n-butyl acetate, fed in over 3 h After addition of the streams, the reaction mixture was stirred at 80° C. for 2 h and then cooled to 25° C.

Inventive:

A reaction vessel was charged under a nitrogen atmosphere with 210 g of n-butyl acetate, 14.7 g of n-butyl acrylate (stabilized with 21 ppm by weight of 5-methyl-2-phenyl-4H-pyrazol-3-one based on the amount of n-butyl acrylate), and 0.08 g of AIBN. The initially charged reaction mixture was heated to 80° C. with stirring. 15 minutes after reaching a temperature of 80° C., two streams were fed in while maintaining the temperature.
Stream 1: 195.3 g of n-butyl acrylate (stabilized with 21 ppm by weight of 5-methyl-2-phenyl-4H-pyrazol-3-one based on the amount of n-butyl acrylate) and 100.5 g of n-butyl acetate, fed in over 2 h
Stream 2: 0.98 g of AIBN and 74.1 g of n-butyl acetate, fed in over 3 h

After addition of the streams, the reaction mixture was stirred at 80° C. for 2 h and then cooled to room temperature.

The polymers obtained were analyzed by GPC (calibration with polystyrene standard, eluent: THF+0.1% by volume of trifluoroacetic acid).

| | Mw (g/mol) |
|---|---|
| Polymer (inventive) | 164 000 |
| Polymer (MEHQ) | 163 000 |

Emulsion Polymerization of n-Butyl Acrylate, Styrene, and Methacrylic Acid:

Comparative Experiment:

A reaction vessel was charged under a nitrogen atmosphere with 250 g of demineralized water and 4 g of potassium peroxodisulfate. The initially charged reaction mixture was heated to 60° C. with stirring. At 60° C., 8 g of feed 1 was fed into the reaction mixture, with stirring. 5 minutes after the addition, the rest of feed 1 and feed 2 were fed into the reaction mixture at 60° C., with stirring. The rest of feed 1 was fed into the reaction mixture over a period of 160 minutes, with feed 2 fed in over a period of 150 minutes.
Feed 1: 4 g of sodium hydrogen sulfite, 100 g of demineralized water
Feed 2: 400 g of styrene (stabilized with 20 ppm by weight of MEHQ based on the amount of styrene), 400 g of n-butyl acrylate (stabilized with 15 ppm by weight of MEHQ based on the amount of n-butyl acrylate), 15 g of methacrylic acid (stabilized with 200 ppm by weight of MEHQ based on the amount of methacrylic acid), 20 g of Disponil FES 147, and 450 g of demineralized water.

At the end of addition of feed 1, the temperature of the reaction mixture was increased to 65° C. and the reaction mixture held at this temperature for 1 h. The dispersion obtained was cooled to ambient temperature, adjusted to pH 7 with aqueous ammonia solution (25% by weight), and filtered through a sieve.

Inventive:

A reaction vessel was charged under a nitrogen atmosphere with 250 g of demineralized water and 4 g of potassium peroxodisulfate. The initially charged reaction mixture was heated to 60° C. with stirring. At 60° C., 8 g of feed 1 was fed into the reaction mixture, with stirring. 5 minutes after the addition, the rest of feed 1 and feed 2 were fed into the reaction mixture at 60° C., with stirring. The rest of feed 1 was fed into the reaction mixture over a period of 160 minutes, with feed 2 fed in over a period of 150 minutes.
Feed 1: 4 g of sodium hydrogen sulfite, 100 g of demineralized water
Feed 2: 400 g of styrene (stabilized with 28 ppm by weight of edaravone based on the amount of styrene), 400 g of n-butyl acrylate (stabilized with 21 ppm by weight of edaravone based on the amount of n-butyl acrylate), 15 g of methacrylic acid (stabilized with 280 ppm by weight of edaravone based on the amount of methacrylic acid), 20 g of Disponil FES 147, and 450 g of demineralized water.

At the end of addition of feed 1, the temperature of the reaction mixture was increased to 65° C. and the reaction mixture held at this temperature for 1 h. The dispersion obtained was cooled to ambient temperature, adjusted to pH 7 with aqueous ammonia solution (25% by weight), and filtered through a sieve.

| | Average | Glass | | | Residual mon- |
|---|---|---|---|---|---|
| | particle | temper- | | Coagulate | omer content |
| | diameter | ature | Mw | (% by | (ppm by |
| | (nm) | (° C.) | (g/mol) | weight) | weight) |
| Comparative | 163 | 19 | 820 000 | 0.25 | n-Butyl |
| experiment | | | | | acrylate: 300 |
| | | | | | Styrene: <20 |
| Inventive | 168 | 21 | 850 000 | 0.4 | n-Butyl |
| | | | | | acrylate: 500 |
| | | | | | Styrene: <20 |

The average particle size (z average) was determined as follows:

The samples were analyzed with a Malvern Zetasizer Nano analyzer. They were diluted with Millipore water (pH 4) to a measurement concentration of 0.0005% and then determined with 5 μm filtration. The measurements were carried out at 25° C.

The glass temperature was determined as follows:

The polymer dispersions were dried under reduced pressure at 80° C. The glass temperature was determined by DSC. Before measurement, the samples were rapidly cooled from 140° C. and then measured at 20 K/min from −50° C. to 140° C.

Molar masses were determined by GPC (calibration with polystyrene standard, eluent: THF+0.1% by volume of trifluoroacetic acid).

The residual monomer content was determined by headspace GC.

The amount of coagulate (aggregated polymer colloids in the apparatus and in the filtration residue) was weighed.

1.-13. (canceled)

14. A mixture comprising one or more compounds of the general formula (II) 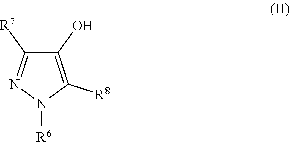
where
R⁶ is H, C₁ to C₂₀ alkyl, where C₁ to C₂₀ alkyl comprises straight-chain or branched C₁ to C₂₀ alkyl groups or cyclic C₃ to C₂₀ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
R⁷ is H, C₁ to C₂₀ alkyl, where C₁ to C₂₀ alkyl comprises straight-chain or branched C₁ to C₂₀ alkyl groups or cyclic C₃ to C₂₀ alkyl groups, phenyl, carboxylic esters having C₁ to C₆ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl, and
R⁸ is H, C₁ to C₂₀ alkyl, where C₁ to C₂₀ alkyl comprises straight-chain or branched C₁ to C₂₀ alkyl groups or cyclic C₃ to C₂₀ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl, and (meth)acrylic acid and/or C₁ to C₈ alkyl (meth)acrylates.

15. The mixture according to claim 14, wherein
R⁶ is H, C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl,
R⁷ is H, C₁ to C₈ alkyl, where C₁ to C₈ alkyl comprises straight-chain or branched C₁ to C₈ alkyl groups or cyclic C₃ to C₈ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl, and
R⁸ is H, C₁ to C₈ alkyl, where C₁ to C₈ alkyl comprises straight-chain or branched C₁ to C₈ alkyl groups or cyclic C₃ to C₈ alkyl groups, phenyl, carboxylic esters having C₁ to C₈ alkyl, where C₁ to C₈ alkyl comprises straight-chain or branched C₁ to C₈ alkyl groups or cyclic C₃ to C₈ alkyl groups, alkanoyl having C₁ to C₈ alkyl, where C₁ to C₈ alkyl comprises straight-chain or branched C₁ to C₈ alkyl groups or cyclic C₃ to C₈ alkyl groups, cyanophenyl, 2-chlorophenyl, 3-chlorophenyl, 4-chlorophenyl, 2,4-dichlorophenyl, 3,4-dichlorophenyl, 2-bromophenyl, 3-bromophenyl, 4-bromophenyl, 2-methylphenyl, 3-methylphenyl, 4-methylphenyl, dimethylphenyl, such as 3,4-dimethylphenyl, 2-methoxyphenyl, 3-methoxyphenyl, 4-methoxyphenyl, 2-ethylphenyl, 3-ethylphenyl, 4-ethylphenyl, 2-ethoxyphenyl, 3-ethoxyphenyl, 4-ethoxyphenyl, 4-isopropylphenyl, benzyl, 2-pyridyl, 3-pyridyl, 4-pyridyl, (carboxymethyl)phenyl, in which the carboxyl group may also be esterified with C₁ to C₆ alkyl, where C₁ to C₆ alkyl comprises straight-chain or branched C₁ to C₆ alkyl groups or cyclic C₃ to C₆ alkyl groups, (N,N-dimethylsulfonamido)phenyl, sulfonamidophenyl or 4-isopropoxyphenyl.

16. The mixture according to claim 14, wherein
R⁶ is H or C₁ to C₆ alkyl, where C₁ to C₆ alkyl comprises straight-chain or branched C₁ to C₆ alkyl groups or cyclic C₃ to C₆ alkyl groups,
R⁷ is H, C₁ to C₆ alkyl, where C₁ to C₆ alkyl comprises straight-chain or branched C₁ to C₆ alkyl groups or cyclic C₃ to C₆ alkyl groups, or alkanoyl having C₁ to C₆ alkyl, where C₁ to C₆ alkyl comprises straight-chain or branched C₁ to C₆ alkyl groups or cyclic C₃ to C₆ alkyl groups, and
R⁸ is H or C₁ to C₆ alkyl, where C₁ to C₆ alkyl comprises straight-chain or branched C₁ to C₆ alkyl groups or cyclic C₃ to C₆ alkyl groups.

17. The mixture according to claim 14, wherein C₁ to C₈ alkyl (meth)acrylates are methyl (meth)acrylate, ethyl (meth)acrylate, n-butyl (meth)acrylate, 2-ethylhexyl (meth)acrylate or a mixture of two or more of said (meth)acrylic esters.

18. The mixture according to claim 14, wherein the total amount of compounds of the general formula (II) in the mixture is 0.1 to 1000 ppm by weight based on the total weight of the amount of (meth)acrylic acid and C₁ to C₈ alkyl (meth)acrylates present in the mixture.

19. The mixture according to claim 14, wherein the total amount of (meth)acrylic acid and C₁ to C₈ alkyl (meth)acrylates in the mixture is at least 5% by weight based on the total weight of the mixture.

20. The mixture according to claim 14, wherein the mixture comprises (meth)acrylic acid and wherein the mixture comprises one or more of the following components:
a. water 20 to 15 000 ppm by weight based on the total weight of (meth)acrylic acid present in the mixture,
b. di(meth)acrylic acid 100 to 20 000 ppm by weight based on the total weight of (meth)acrylic acid present in the mixture,
c. acetic acid 100 to 2000 ppm by weight based on the total weight of (meth)acrylic acid present in the mixture,
d. propionic acid 100 to 2000 ppm by weight based on the total weight of (meth)acrylic acid present in the mixture,
e. one or more aldehydes, such as acetaldehyde, acrolein, propionaldehyde, 2-furfural, 3-furfural, coumarone or benzaldehyde, the amount of each aldehyde being 0 to 1 ppm by weight based on the total weight of (meth)acrylic acid present in the mixture,
f. protoanemonin 0 to 2 ppm by weight based on the total weight of (meth)acrylic acid present in the mixture,
g. maleic acid 0 to 1 ppm by weight based on the total weight of (meth)acrylic acid present in the mixture,
h. maleic anhydride 0 to 1 ppm by weight based on the total weight of (meth)acrylic acid present in the mixture,
i. allyl acrylate 0 to 30 ppm by weight, preferably 0 to 1 ppm by weight, based on the total weight of (meth)acrylic acid present in the mixture.

21. The mixture according to claim 17, wherein the mixture comprises one or more (meth)acrylic esters according to claim 17 and wherein the mixture comprises one or more of the following components:
a. water 1 to 2000 ppm by weight, preferably 1 to 500 ppm by weight, in each case based on the total weight of (meth)acrylic esters according to claim 17 present in the mixture,
b. (meth)acrylic acid 1 to 2000 ppm by weight, preferably 1 to 100 ppm by weight, in each case based on the total weight of (meth)acrylic esters according to claim 17 present in the mixture,
c. alcohols of the respective (meth)acrylic esters 0.01 to 2000 ppm by weight, preferably 0.1 to 1000 ppm by weight, in each case based on the total weight of (meth)acrylic esters according to claim 17 present in the mixture,
d. propionic esters with the alcohols of the respective (meth)acrylic esters 0.1 to 2000 ppm by weight, preferably 10 to 1000 ppm by weight, in each case based on the total weight of (meth)acrylic esters according to claim 17 present in the mixture,
e. acetic esters with the alcohols of the respective (meth)acrylic esters 1 to 2000 ppm by weight, preferably 5 to 2000 ppm by weight, in each case based on the total weight of (meth)acrylic esters according to claim 17 present in the mixture,
f. ethers of the respective (meth)acrylic ester alcohols 0 to 2000 ppm by weight, in each case based on the total weight of (meth)acrylic esters according to claim 17 present in the mixture.

22. The mixture according to claim 14, wherein the mixture is stored and/or transported under an oxygen-containing atmosphere.

23. The mixture according to claim 14, wherein the mixture comprises a co-stabilizer.

24. The use of one or more compounds of the general formula (II) as defined in claim 14 for the storage- and/or transport-stabilization of (meth)acrylic acid and/or C₁ to C₈ alkyl (meth)acrylates.

25. A method for polymerizing (meth)acrylic acid and/or C₁ to C₈ alkyl (meth)acrylates in the presence of one or more compounds of the general formula (II) as defined in claim 14, the reaction mixture being 0 to 50% saturated with oxygen, the degree of oxygen saturation referring to the degree of oxygen saturation that is achieved at equilibrium when the mixture is stored and/or transported under standard conditions under an oxygen-containing atmosphere having an oxygen content of 5 to 30 vol %.

26. A method for stabilizing (meth)acrylic acid and/or C₁ to C₈ alkyl (meth)acrylates through the use of one or more compounds of the general formula (II) as defined in claim 14.
